(19) United States
(12) Patent Application Publication
(10) Pub. No.: US 2006/0040280 A1
(43) Pub. Date: Feb. 23, 2006

(54) Functionalized materials and methods of using same

(76) Inventors: William Lee, Cambridge, MA (US); Saito Kyoichi, Tokyo (JP)

Correspondence Address: FOLEY & LARDNER LLP, 111 HUNTINGTON AVENUE, 26TH FLOOR, BOSTON, MA, 02199-7610, US

(21) Appl. No.: 11/085,023

(22) Filed: Mar. 15, 2005

Related U.S. Application Data
(63) Continuation of application No. PCT/US03/29448, filed on Sep. 18, 2003.
(60) Provisional application No. 60/411,659, filed on Sep. 18, 2002.

(51) Int. Cl. C12Q 1/68; C12M 1/34

(52) U.S. Cl. 435/6; 435/287.2

(57) ABSTRACT

Compositions are provided herein comprising a base material having engrafted polymer brushes. The polymer brushers further comprise one or more functional groups immobilized along the surface of the brushes in a plurality of layers, which confer functional properties to the base material compositions. Methods of using these compositions include deoxygenation of a sample solution, hydrolysis of denaturing agents in a sample solution, resolution of racemic mixtures in a sample solution, and purification, and concentration of target compounds.

### FIELD OF THE INVENTION

The present invention relates to materials for the separation, purification, concentration, immobilization and synthesis of compounds, as well as applications for using the same.

### BACKGROUND OF THE INVENTION

Isolation and purification of a target molecule is a prerequisite to its study and use, for example, the ability to isolate and identify disease causing microorganisms allows for accurate diagnosis and treatment of disease states, or isolation of a nucleic acid is the first step in the sequencing of the polynucleotide or the polypeptide sequence encoded by a nucleic acid, or the determination of the crystal structure of a protein. There are many methods for isolating, purifying, and concentrating molecules, but the compositions for performing such methods do not have broad application, and are usually applicable to the purification of specific molecules. There remains a need in the art for improved compositions and methods of isolating and concentrating molecules.

### SUMMARY OF THE INVENTION

In general, the invention is based on the discovery that certain materials can be fabricated into compositions that have side chains or polymeric molecular “brushes” which have particular properties, for example, length, thickness, morphology and density. The materials are highly effective for separating, purifying, concentrating and/or immobilizing compounds in a three dimensional conformation, and for synthesizing or otherwise modifying compounds immobilized thereto. The compositions of the present invention are useful in applications that require a high convective flow rate across the material, or are subjected to harsh chemicals, or extreme temperature variations.

In one embodiment, the invention provides for compositions which comprise one or more base materials having defined shapes or textures. The base materials further comprise polymeric brushes having one or more functional groups immobilized thereto. In another embodiment the base material has a plurality of surfaces, which define at least one lumenal space. In one aspect these lumenal spaces comprise pores. In yet another aspect these lumenal spaces comprise channels. In one aspect, the functional groups are anionic dissociating functional groups. In another aspect, the functional groups are cation dissociating functional groups. In yet another aspect, the functional groups are anionic dissociating and cation dissociating functional groups. In still another aspect, the functional groups are polypeptides, for example, enzymes, antibodies, cellular receptors, affinity purification epitopes, and fragments or active domains of the same. In another aspect, the functional groups are nucleic acids or chemically modified variants thereof, for example, deoxyribonucleic acid, ribonucleic acid, polyA+ RNA, tRNA, rRNA, aptamers or ribozymes. In still another aspect, the functional groups are polypeptide functional groups, nucleic acid functional groups, ionic functional groups, hydrophilic functional groups, or any such combination thereof. In yet another aspect, multiple functional groups are immobilized, for example, a first functional group is immobilized by the polymer brush and a second functional group is immobilized by the first functional group, or the first functional group immobilizes both a second or third functional group.

The invention provides for high capacity adsorption of functional groups to the polymer brushes of the base material compositions. In one embodiment, the functional groups are immobilized in multiple layers along the polymeric side chain brushes. In one aspect, the functional groups are immobilized along the longitudinal surface of a polymer brush in multi-layers, for example 50 layers. In one aspect, the brushes themselves provide for physical retention of the functional groups. In another aspect, functional groups are immobilized by ionic interaction with the brush surface. In yet another aspect, the functional groups are covalently attached to the brush surface, for example, the functional groups are cross-linked to the polymer brushes, or a first functional group is crosslinked to a second functional group or a third functional group.

The compositions of the present invention can be incorporated into a variety of products and processes useful in biotechnological, pharmaceutical and chemical applications, to impart desirable properties to these products and processes. In one aspect of the invention, the compositions described herein are used as a high capacity matrix for concentration, separation and purification applications. In another aspect, the compositions are used as containers for storing or transferring solutions. In one aspect the container is a functionalized pipet tip comprising polymer brushes, said polymer brushes further comprising one or more functional groups immobilized on the surface of said polymer brushes in a plurality of layers. In another aspect the container is a tube comprising polymer brushes, said polymer brushes further comprising one or more functional groups immobilized on the surface of said polymer brushes in a plurality of layers. In these aspects, the container possesses a functional property determined, i.e., by the properties of the brush and the functional group immobilized thereto, examples of containers are, such as but not limited to, a pipet tip or tube comprising affinity purification functional groups used in separation applications, or ion exchange functional groups for the removal of nucleic acids from cellular lysates, or a freezing vial comprising cryopreservative functional groups is used for the storage of samples, or tubing. In another aspect, the compositions provide surfaces for the synthesis of polynucleotides or polypeptides. In yet another aspect, the compositions provide functional groups having an affinity for a compound, and chemical or biological modifications to the compound can be made directly to the immobilized compound.

The invention provides compositions and methods with a wide range of applications, for example, in high throughput screens for proteomics and genomics applications, peptide synthesis applications, combinatorial chemistry applications, nucleic acid synthesis applications, in the production of chemical or pharmaceutical compositions, in bioremediation applications, in microbiology applications, in diagnostic applications, and in dialysis or filtration applications. In one aspect, a DEA or positively charged membrane removes nucleic acids in protein purification applications.

In one embodiment, the invention provides compositions comprising at least one base material further comprising polymer brushes, said polymer brushes further comprising one or more functional groups immobilized on the surface of said polymer brushes in a plurality of layers, wherein said functional groups react with a substrate compound when contacted with said substrate compound. In one aspect the reaction consists of immobilization of the substrate compound to the polymer brushes. In another aspect the reaction consists of hydrolysis of the substrate compound. In yet another aspect the reaction consists of deoxygenation of the substrate compound. In still another aspect the reaction consists of polymerization, synthesis, or modification of the substrate compound.

In one embodiment, the invention provides compositions and methods for adsorbing and/or immobilizing target compounds from liquid solutions. The method includes the steps of obtaining a base material, engrafting polymeric brushes thereto, immobilizing functional groups to the brushes, optionally immobilizing a second functional group to the first functional group, or a third functional group to the first or second functional group, and contacting the brushes with a sample solution containing a target compound having affinity for one or more of the immobilized functional groups, thereby adsorbing the compound. In one aspect, the functional groups are cross-linked to other functional group, and as such, to the brushes. This prevents detachment of the functional groups from the brush, induced by changes in such variables as, for example eluent, pressure, pH, ionic strength, solvent type and concentration, and temperature.

In one embodiment, the present invention provides methods and compositions for deoxygenating a substrate compound, comprising obtaining a base material having polymer brushes grafted to said base material, wherein said polymer brushes further comprise at least one functional group immobilized in a plurality of layers to the surface of said polymer brushes, and contacting the base material with said substrate compound, thereby deoxygenating the substrate compound. In this aspect, a base material is obtained, and brushes are grafted thereon, the brushes having deoxygenating functional groups, for example, ascorbic acid oxidase (AsOM). In one aspect, the functional groups are immobilized in multi-layers on the polymer brushes. The base material is contacted with the sample solution having the target compound, ascorbic acid (AsA), in the above example. Quantitative conversion of AsA into dehydroascorbic acid is monitored to determine the rate and extent of deoxygenation of the AsA in the sample solution.

In another embodiment, the invention provides compositions and methods for asymmetrically hydrolyzing a substrate compound further comprising a racemic mixture, comprising obtaining a base material having polymer brushes grafted to said base material, wherein said polymer brushes further comprise at least one functional group immobilized in a plurality of layers to the surface of said polymer brushes, and contacting the base material with said substrate compound, thereby asymmetrically hydrolyzing the racemic mixture. For example, the functional group aminoacylace is immobilized to the polymer brushes, and the base material is contacted with a racemic amino acid mixture, i.e., an acetyl-DL-methionine solution. In this example the production of L-methionine is monitored to determine the rate and extent of hydrolysis of racemic mixtures in the sample solution.

In another embodiment, the invention provides compositions and methods hydrolyzing a substrate compound further comprising a denaturing agent, comprising obtaining a base material having polymer brushes grafted to said base material, wherein said polymer brushes further comprise at least one functional group immobilized in a plurality of layers to the surface of said polymer brushes, and contacting the base material with said substrate compound, thereby hydrolyzing the denaturing agent. In one aspect the denaturing agent is urea and the functional group is the enzyme urease.

In another embodiment, the invention provides a method for conditioning the polymer brushes prior to immobilization of functional groups to modulate multi-layering of the functional groups on the brush surfaces. A base material is obtained having polymer brushes, said polymer brushes having, for example, anionically dissociating first functional groups, cationically dissociating second functional groups and hydrophilic third functional groups immobilized thereto, The base material is treated with a acid thereby modulating the conformation of said polymer brushes, and a fourth functional group is immobilized in a plurality of layers to said polymer brushes. The base material is treated with an alkali thereby modulating the conformation of said polymer brushes, and a fifth functional group is immobilized in a plurality of layers to said polymer brushes. The order of treating with an acid and an alkali can be reversed.

The base material comprising a plurality of polymer brushes is conditioned, for example with an acid such as hydrochloric acid, before the immobilization of functional groups. In this aspect, the base material exhibits a high degree of multi-layering, i.e., immobilization of functional groups along the longitudinal surface of a polymer brush. The conditioning permits the polymer brushes to extend or contract, thus varying the degree and type of functional group multi-layering on the brushes, for example, the brushes are contracted before a first functional group is immobilized thereto, and expanded before a second functional group is immobilized thereto, thus providing a brush surface comprising two functional groups in substantially discrete multilayers along the longitudinal surface. Alkaline solutions are used to expand polymer brushes comprising cation dissociating functional groups and contract polymer brushes comprising anion dissociating functional groups, while acidic solutions are used to expand polymer brushes comprising anion dissociating functional groups and contract polymer brushes comprising cation dissociating functional groups. Thus conditioning provides for modulating the multi-layering of one or more functional groups on the brush surface.

In one embodiment the invention provides a base material comprising polymer brushes having one or more functional groups immobilized thereto manufactured by the steps comprising obtaining a base material further having polymer brushes, said polymer brushes further comprising ionically dissociating groups and hydrophilic groups, treating said base material with an ionic solution thereby modulating the conformation of said polymer brushes, and immobilizing one or more functional groups to the surface of said polymer brushes in a plurality of layers.

In still another embodiment of the invention, the invention provides for methods of enhancing immobilization of functional groups to the polymer brushes, by cross-linking to the polymer brushes, for example, cross-linking via glutaraldehyde treatment. In one aspect, the functional groups are cross-linked in multi-layers.

The invention provides a tube or conduit having an interior surface and an exterior surface, the interior surface defining a lumenal space, and the interior surface having grafted polymer brushes further having functional groups disposed along the surface of the brush. In various embodiments, the functional groups are disposed along the brush surface in a plurality of layers and include hydrophilic groups, hydrophobic groups, anionic groups cationic groups, affinity groups, enzyme groups and any combinations thereof. A solution is passed through the lumen of the conduit and molecules in the solution are adsorbed to the functional groups thereby reducing the concentration of the molecules in the solution as the solution is passed through the conduit. In another embodiment, a solution is passed through the lumen of the conduit and molecules in the solution react with the functional groups thereby reacting with the molecules in the solution as the solution is passed through the conduit. In another embodiment, the conduit provides for a method of using the conduit to remove or separate nucleic acids or polypeptides from the solution as well as a method for purifying these from the solution.

The invention also provides a method of making a functionalized conduit by obtaining a conduit having an interior surface and an exterior surface, the interior surface defining a lumenal space, grafting polymer brushes to the interior surface, and immobilizing functional groups on the surface of the brushes.

Other features and advantages of the invention will be apparent from following detailed description, and from the claims.

### DETAILED DESCRIPTION OF THE INVENTION

Definitions

Unless otherwise defined, all technical and scientific terms used herein have the same meaning as commonly understood by one of ordinary skill in the art of which this invention belongs. However, the following terms have the meanings specified below.

As used herein, the term “base material” refers to a substrate providing one or more surfaces, where the surface is capable of forming polymer brushes, or to which polymer brushes can be grafted or otherwise affixed. The form of the base material may be substantially rigid, for example, a vial, a pipet tip, a cell culture or ELISA dish, slide or array, or the base material may be substantially flexible along one or more planes, for example a fiber or membrane, or the base material may be in the form of a powder or microcrystaline preparation. The base material may be substantiating elongated and flexible, and may define a lumen, i.e., as in conduit or tubing for example. A wide variety of base materials are appropriate for the membrane compositions and methods disclosed herein, and are described below and in U.S. Pat. Nos. 6,009,739, 5,783,608, 5,743,940, 5,738,775, 5,648,400, 5,641,482, 5,506,188, 5,425,866, 5,364,638, 5,344,560, 5,308,467, 5,075,342, 5,071,880, 5,064,866, 4,980,335, 4,897,433, 4,622,366, 4,539,277, 4,407,846, 4,379,200, 4,376,794, 4,288,467, 4,287,272, 4,283,442, 4,273,840, 4,137,137 and 4,129,617, each incorporated herein by reference.

As used herein, the term “brush” or “polymer brush” refers to a polymeric side chain that is formed from a polymerization substrate having a radical-polymerizable terminal group, wherein the polymerizable substrate is the base material, or can be engrafted to or otherwise affixed to the base material, thereby substantially taking the form of the base material. The side chain can be any polymer, but an easily functionalizable reactive polyvinyl polymer is currently preferred, for example such as poly(glycidyl methacrylate), which has one reactive epoxide group per repeat. Polymer brushes are formed by radical polymerization as described below. A brush has an elongated shape of a particular size in one direction related to the degree of polymerization in a first direction, its “length”, and a cross sectional diameter or thickness related to the degree of polymerization in a second direction perpendicular to the first direction, its “width”. The brushes can assume a coiled or compacted morphology or an extended morphology. The width of a brush can vary along its length. In addition, the polymerization reaction can be controlled to create branch-like polymer brush structures, as well as increasing or decreasing brush density, i.e., number of brushers per surface area or per weight of base material, as described below. The length, width, branching, and overall morphology of the polymer brushes in the present invention can be varied according to the desired end use or purpose as described herein and by methods known in the art.

As used herein the term “reactive monomer” refers to a compound that is capable of participating in a radical induced grafting reaction. The reactive monomer can be any material capable of forming polymers as described above and herein, for example but not limited to glycidyl methacrylate (GMA), or ethylene. The base material and reactive monomer may be of the same compound, for example, a polyethylene base material may utilize ethelyene monomers or polymers in the grafting reaction. A wide variety of reactive monomers are appropriate for the membrane compositions and methods disclosed herein, and are described below and in U.S. Pat. Nos. 6,009,739. 5,783,608, 5,743,940, 5,738,775, 5,648,400, 5,641,482, 5,506,188, 5,425,866, 5,364,638, 5,344,560, 5,308,467, 5,075,342, 5,071,880, 5,064,866, 4,980,335, 4,897,433, 4,622,366, 4,539,277, 4,407,846, 4,379,200, 4,376,794, 4,288,467, 4,287,272, 4,283,442, 4,273,840, 4,137,137 and 4,129,617, each incorporated herein by reference.

As used herein the term “degree of polymerization” refers to the extent of radical induced polymerization of a polymerizable substrate having a radical-polymerizable terminal group, with a reactive monomer, wherein said polymerization reaction forms a polymer brush. The degree of polymerization is thus determinative of the overall brush surface characteristics. The polymeric side chains can, for example, be a monomer, an oligomer, or have an average length between about 10 nm and about 2000 nm corresponding to anywhere from about several hundred to tens of thousands of monomer units or longer, for example about 5000 nm or more. The degree of polymerization depends on, e.g., the crystalinity of the polymerizable substrate, the degree of radicalization, the length of time the reaction is allowed to progress, and on the physical properties of the polymerizable substrate, i.e., its strength or rigidity (see, Lee, et al., (1999) Chem, Mater., 11, 3091-3095, incorporated herein by reference).

As used herein the term “degree of grafting” or “DG” refers to the brush density, i.e., the number of the side chains brushes per unit surface area of base material. Anywhere from about 1.0×10⁸ to about 1.0×10³⁰ of the side chains brushes can be present per square meter of surface area or per weight of base material, for example, from about 1.0×10¹⁶ to about 1.0×10²⁰ of the side chains brushes represents a degree of grafting between about 10% and about 500%. The degree of grafting is essentially a ratio describing the initial weight of a base material and the additional weight of the polymer brush structures (see, Lee, et al., (1999).

As used herein a “functional group” refers to a compound having a particular chemical property, biological activity or affinity for a ligand, or a particular structure. A functional group is immobilized, bound, entrapped, cross-linked or otherwise substantially affixed to the polymer brushes grafted to the base material. A wide variety of functional groups are suitable for the present membrane compositions and methods, imparting such functionality to the brushes. Combinations of functional groups are clearly within the scope of the invention. Enzymes are also desirable as functional groups, for example, restriction endonucleases, proteases, anticlotting factors and anticoagulants such as anti-thrombin, and other enzymes that can impart functionality to the brushes. Suitable functional groups include, for example and without limitation, anionically dissociating groups (e.g., primary, secondary, tertiary, or quaternary amines), cationically dissociating groups (e.g., acid groups) with or without coexisting hydrophilic or hydrophobic groups (nonionic groups such as, GMA or other hydrophobic reactive groups), polypeptides, polynucleotides, proteins or active domains thereof, epitopes ions and affinity tags, nucleic acids, ribonucleic acids, polypeptides, glycopolypeptides, mucopolysaccharides, lipoproteins, lipopolysaccharides, carbohydrates, enzymes or co-enzymes, hormones, chemokines, lymphokines, antibodies, ribozymes, aptamers, interferon, SpA, SpG, TNF, v-Ras, c-Ras, reverse transcriptase, G-coupled protein receptors (GPCR's), FcRn, FcγR's, FcεR's, nicotinicoid receptors (nicotinic receptor, GABA_{A} and GABA_{C} receptors, glycine receptors, 5-HT₃ receptors and some glutamate activated anionic channels), ATP-gated channels (also referred to as the P2X purinoceptors), glutamate activated cationic channels (NMDA receptors, AMPA receptors, Kainate receptors, etc.), hemagglutinin (HA), receptor-tyrosine kinases (RTK's) such as EGF, PDGF, NGF and insulin receptor tyrosine kinases, SH2-domain proteins, PLC-γ, c-Ras-associated GTPase activating protein (RasGAP), phosphatidylinositol-3-kinase (PI-3K) and protein phosphatase 1C (PTP1 C), as well as intracellular protein tyrosine kinases (PTK's), such as the Src family of tyrosine kinases, glutamate activated cationic channels (NMDA receptors, AMPA receptors, Kainate receptors, etc.), protein-tyrosine phosphatases, such as receptor tyrosine phosphatase rho, protein tyrosine phosphatase receptor J, receptor-type tyrosine phosphatase D30, protein tyrosine phosphatase receptor type C polypeptide associated protein, protein tyrosine phosphatase receptor-type T, receptor tyrosine phosphatase gamma, leukocyte-associated Ig-like receptor 1D isoform, LAIR-1D, LAIR-1C, MAP kinases, neuramimidase (NA), proteases, polymerases, serine/threonine kinases, second messengers, antigenic or tumorigenic markers, transcription factors, and other such important metabolic building blocks or regulators. Selection and use of functional groups is described below and in U.S. Pat. Nos. 6,009,739, 5,783,608, 5,743,940, 5,738,775, 5,648,400, 5,641,482, 5,506,188, 5,425,866, 5,364,638, 5,344,560, 5,308,467, 5,075,342, 5,071,880, 5,064,866, 4,980,335, 4,897,433, 4,622,366, 4,539,277, 4,407,846, 4,379,200, 4,376,794, 4,288,467, 4,287,272, 4,283,442, 4,273,840, 4,137,137 and 4,129,617, each incorporated herein by reference.

The term “anionically dissociating functional groups” as used herein means those ion-exchange groups whose counter ion is an anion. Anionically dissociating groups have the ability catalyze chemical reactions and to absorb and/or immobilize target compounds or other functional groups and are capable of entering into neutralizing reactions with acidic substances such as hydrogen sulfide or mercaptans, allowing for a wide range of uses with effective removal of the acidic substances.

The term “cationically dissociating functional groups” as used herein means those ion-exchange groups whose counter ion is a cation. A typical cationically dissociating group is an acid group. Cationically dissociating groups have the ability to catalyze chemical reactions and adsorb and/or immobilize target compounds or other functional groups and are capable of releasing a proton (hydrogen ion) to enter into neutralizing reaction with basic substances, say, ammonia or amines. As a result, these groups provide a wide range of uses with basic substances.

The term “hydrophilic functional groups” as used herein refers to groups that have an affinity for water but do not undergo significant ionic dissociation upon contact with water. Hydrophilic groups have the ability to catalyze chemical reactions and adsorb and/or immobilize target compounds or other functional groups, by providing a hydration shell, or by providing a reactive surface. An example of such group, without limitation, is a hydroxyl group, such as those found on hydroxyethyl methacrylate (HEMA).

The term “hydrophobic functional groups” as used herein refers to groups that do not have an affinity for water. Hydrophobic groups have the ability to catalyze chemical reactions and adsorb and/or immobilize target compounds or other functional groups, by excluding water, or by providing a surface for hydrophobic interactions, or by providing a reactive surface. An example of such group, without limitation, is a nonionic group, an ester group, a succinimide group or an epoxy group.

Detailed Description of the Invention

The present invention provides for compositions and methods of immobilizing functional groups to polymer brushes grafted to one or more base materials. Immobilization methods include entrapment, gelification, physical retention or adsorption, ionic binding, covalent binding or cross-linking (see, Biotechnol. Bioeng., 22:735-756, 1980; Chem. Eng. Prog., 86:81-89, 1990; J. Am. Chem. Soc., 117:2732-2737, 1995; Enzyme Microb. Technol., 14:426-446, 1997; Trends Biotechnol., 13:468473, 1997; Nat. Biotechnol., 15:789-793, 1997, each incorporated herein by reference). The immobilization method and the amount and kind of the functional groups used both determine the activity of the composition of the present invention. The resulting activity of the immobilized functional group can often further reduced by mass-transfer effects (see, Methods Enzymol., 44:397-413, 1976; J. Am. Chem. Soc., 114:7314-7316, 1992; Trends Biotechnol., 14:223-229, 1996; Angew. Chem., 109:746-748, 1997, each incorporated herein by reference). The activity following immobilization can be further reduced as a result of the diminished availability of the functional groups, i.e., due to steric hindrance, entrapment within brushes, pores or other structures on the base material substrate, or by slow diffusion of the functional groups. Such limitations lead to lowered efficiency. It is an objective of the present invention to provide base materials having a high capacity for functional groups immobilized thereto,

The invention is usable with a wide variety of base materials, i.e., all polymeric plastics, such as, for example, polyurethanes, polyamides, polyesters, polyethers, polyether-block amides, polystyrene, polyvinyl chloride, polycarbonates, polyorganosiloxanes, polyolefins, polysulfones, polyisoprene, polychloroprene, polytetrafluoroethylene (PTFE), corresponding copolymers and blends, as well as natural and synthetic rubbers, metal, glass or wooden bodies. The compositions have multifunctional properties and can be used to separate, remove, purify, synthesize, concentrate and immobilize compounds, and are particularly suited to the harsh operating environments, i.e., extreme temperatures and pressures, chemical concentrations, electrical charges, etc., from commercial processes. In general, the desired target compound is in a sample solution, which can be passed directly through the compositions, as in a filtration membrane, tube, pipet tip or a chromatography matrix. Liquids containing cells or other large insoluble particles may require pre-treatment to separate the larger particles from the smaller soluble ones. However, the polymer brush sizes and brush density provide a degree of physical filtration, and the compositions can be woven or otherwise fabricated into filtration devices if appropriate. While an aqueous sample solution is often described, one skilled in the art will realize that gaseous samples may be employed. Examples of filter elements for adsorbing gaseous components of a gas stream are described in, for example, U.S. Patent Application 20020002904 A1, to Gentilcore, et al., published Jan. 10, 2002, herein incorporated by reference. In addition, a membrane or fiber is often described, but the compositions of the invention illustrated below can comprise other forms as described herein. Thus the following is illustrative and are not meant to be limiting examples of the present invention.

Materials Useful in the Present Invention

In general, the base material of the present invention is not limited to any particular type, and any substrate that permits grafting or affixation of the polymer brush is an appropriate base material. Treatment of a base material surface is acceptable if the original base material is not sufficient itself, for the polymerization reaction. In such cases, the surface treatment according to the invention can be, for example, a coating formed from a polymeric material. Materials useful in the present invention are widely available, for example polyolefins (low density or high density) including polyethylene and polypropylene, cellulose (see, Radiat. Phys. Chem. 1990, 36:581; J. Membr. Sci. 1993, 85:71), poly(isobutylene oxide) (see, Radiat. Phys. Chem. 1987, 30:151), ethylene-tetrafluoroethylene copolymer (see, J. Electrochem. Soc. 1996, 143: 2795) ethylene-propylene-diene terpolymer (see, Radiat. Phys. Chem. 1991, 37: 83) ethylene-propylene rubber (see, Nippon Gensiryoku Gakkaishi, 1977, 19:340) chlorosulfonated polyethylene (see, Radiat. Phys. Chem. 1991, 37:83) polytetrafluoroethylene (PTFE) (see, React. Polym. 1993, 21:187; Radiat. Phys. Chem. 1989, 33:539) tetrafluroethylene-hexafluoropropylene copolymer (see, Radiat. Phys. Chem. 1988, 32:193) poly(vinyl chloride) (see, Radiat. Phys. Chem. 1978, 11:327) silicone rubber (see, Radiat. Phys. Chem. 1988, 32: 605) polyurethanes (see, Radiat. Phys. Chem. 1981, 18: 323) polyesters (see, Radiat. Phys. Chem. 1988, 31: 579) butadiene-styrene copolymer (see, Radiat. Phys. Chem. 1990, 35: 132) natural and nitrile rubbers (see, Radiat. Phys. Chem. 1989, 33: 87) cellulose acetate and propionate (see, Radiat. Phys. Chem. 1990, 36: 581) starch and cotton fabric (see, Zhum, Vsesoyuz. Khim. Ob-va im. D. I. Mendeleeva. 1981, 26:401) polyester-cellulose fabric (see, Radiat. Phys. Chem. 1981, 18:253) natural leather (see, Radiat. Phys. Chem. 1980, 16:411) and medical gauze (see, Zhum. Vsesoyuz. Khim. Ob-va im. D. I. Mendeleeva. 1981, 26:401) hydrophilic polyurethanes, polyureas, olefins, acrylics, as well as other hydrophilic components. Particular materials include polyethylene glycol, polyethylene glycol or polypropylene glycol copolymers and other poloxamers, heterocyclic monomers (see, Applied Radiation Chemistry: Radiation Processing, Robert J. Woods and Alexei K. Pikaev, John Wiley & Sons, Inc., 1994 (ISBN 0-471-54452-3)), poly(ethylene glycol) methacrylate or dimethacrylate (see, J. Appl. Polym. Sci., 1996, 61:2373-2382), polyamine (such as polyethyleneimine), poly(ethylene oxide), and styrene. These coatings preferably are covalently bonded to the surface which is being treated. Many methods for forming the coating exist, and include the steps of adsorbing the polymeric material to the surface, and then covalently attaching the polymeric material to the surface by exposure to UV radiation, RF energy, heat, X-ray radiation, gamma radiation, electron beams, chemical initiated polymerization or the like.

A base material provides a plurality of surfaces, and may be itself a polymerizable substrate having a radical-polymerizable terminal group, for example, celluloses, polyolefins, polyacrylonitriles, polyesters such as PET and PBT, polyamides such as nylon 6 and nylon 66, as well as combinations of these. An appropriate base material may not be polymerizable itself, provided polymer brushes can be grafted, affixed, or otherwise adhered to the non-polymerizable base material.

A carbohydrate polymer, such as cellulose or lignin, or a similar material, can be used as the base material. An example of a composition and method of a grafted carbohydrate polymer having pendant 3-amino-2-hydroxy propyl groups grafted thereon, for use as a retention aid and strengthening additive in paper manufacture is described in U.S. Patent Application 20020026992 A1, to Antal, et al., published Mar. 7, 2002, incorporated herein by reference. The method of radiation induced grafting to cellulose is described in, Yamagishi et al., (1993) J. Membr. Sci., 85, 71-80, incorporated herein by reference.

When the carbohydrate polymer is a component of wood pulp the resulting chemically modified wood pulp may be employed in conjunction with unmodified wood pulp to incorporate therein the retention and strengthening characteristics. Typical sources of the carbohydrates, specifically celluloses that can be used as the base material include wood celluloses such as paper pulp and wood chips. In addition to these celluloses, leaf fiber cellulose, stem fiber cellulose and seed tomentous or pubescent fiber cellulose can also be used. Examples of such celluloses include bast fibers (e.g., hemp, flax, ramie and Manila hemp) and cotton. If desired, rice straw, coffee bean husk, spent tea leaves, soy pulp and other waste can be recycled for use as cellulose. Such waste is very convenient to use as a base material because it does not require any special preliminary treatments. One such source for cellulose for use in the present invention is paper pulp.

Metallic base materials can be grafted with biologically active compounds, for example surface-modified medical metallic materials having a gold or silver thin layer plated onto a base metal, as described in U.S. Patent Application 20010037144, A1 to Kim, et al., published Nov. 1, 2001 and incorporated herein by reference.

Animal tissues such as fiber, hair, and leather can be used as the base material. One skilled in the art would be able to determine if an animal product provided the desired properties for use as a base material. For example, where it is desired that the invention be used in a mechanical filtration, fibers, for example, can be woven or otherwise fabricated into among other forms, membrane compositions or sheets. Examples of fibers or animal hairs that can be used as base materials include wool, camel hair, alpaca, cashmere, mohair, goat hair, rabbit hair, and silk. Examples of natural leather that can be used as base materials include cowskin, goatskin, and the skin or hide of reptiles. Examples of synthetic leather that can be used as base materials include CORFAM® (DuPont), CLARINO® (Kuraray), and ECSAINE® (Toray).

Polyolefins can also be used as base materials (see, Applied Radiation Chemistry: Radiation Processing, Robert J. Woods and Alexei K. Pikaev, John Wiley & Sons, Inc., 1994 (ISBN 0471-54452-3), Introduction to Radiation Chemistry 3^{rd} Edition, J. W. T. Spinks and R. J. Woods, John Wiley & Sons, Inc., 1990 (ISBN 0471-61403-3), Radiation Chemistry of Polymeric Systems, A. Chapiro, Interscience, New York, 1962, Atomic Radiation and Polymers, A Charlesby, Pergamon Press, 1960, Radiat. Phys. Chem. 1991, 37:175-192, and Prog. Polym. Sci. 2000, 25:371-401 (all incorporated herein by reference in their entirety). Polyolefins can be fabricated into many shapes and forms. They are capable of being molded, thermoformed, poured, extruded and otherwise shaped by processes well known in the art, such as the formation of fibers or filaments by conventional melt spinning processes. In addition, polyolefin compounds are useful in among other industries, the biotechnology industry, largely because polyolefin products are resistant to chemical degradation from common laboratory reagents, are durable and can be reused, and are chemically inert, and are inexpensive and often disposable. Polyolefin compounds are currently preferred base materials as they demonstrate these properties and additionally provide a polymerizable substrate having a radical-polymerizable terminal group. Olefin monomers and polymers are well suited to the grafting techniques of the invention both as base materials and additionally as reactive monomers. Examples of polyolefins include, for example, polyethylene and polypropylene. If desired, these materials can be modified, for example by incorporating halogens into the polymer, such as chlorine, fluorine, or bromine, for example the halogenated polyolefin, polytetraflurorethylene. Other modifications such as incorporation of hydroxyl groups into the polymer are also appropriate. Polyolefinic polymers having weight-averaged molecular weights in the range of from 20,000 to 750,000 daltons are suitable for the present invention. One skilled in the art would know which molecular weights are appropriate for the particular purpose. For example, a polyolefin having a molecular weight from about 50,000 daltons to about 500,000 daltons is suitable to use in the production of fiber or filament, used for example, in a membrane comprising polyolefin filaments or fibers (see, above) further comprising brushes having combinations of functional groups affixed thereto. When the molecular weight of a polyolefin is greater than about 500,000 daltons, the fluidity of the resultant polyolefin is low, and it is difficult to form the polyolefin into such a filament by conventional melt spinning processes. However, the structural rigidity of a polyolefin greater than about 500,000 daltons is suitable, for example, in high density applications such as containers, freezing vials for cells, and the like. By contrast, when the molecular weight of a polyolefin is lower than about 50,000 daltons, the strength and rigidity of the polymer is lessened and a filament obtained therefrom does not have a sufficient tensile strength. However the structural rigidity of a polyolefin when the molecular weight of a polyolefin is lower than about 50,000 daltons is suitable, for example, in a powder or microcrystaline composition. An example of a polymerized grafted and crosslinkable thermoplastic polyolefin powder composition in the form of a powder intended for the production of flexible coatings by its free flow over a hot mold is described in U.S. Patent Application 20020019487 A1, to Valligy, et al., published Feb. 14, 2002, hereby incorporated by reference. Another polymerized grafted and crosslinkable thermoplastic polyolefin powder composition is described in EP0409992, incorporated by reference, is directed to a process for the preparation of particles of crosslinkable thermoplastic polyolefin polymers according to which said particles are brought into contact, in the solid state, with the crosslinking agent, in particular by means of a mineral oil.

The shape of the base material is not limited in any particular way, and various shapes can be employed as selected from among fibers, films, flakes, powders, sheets, mats and spheres. The base material of the membrane of the present invention has the function of serving as a structural member that supports the polymer brushes. The form of the base material may be substantially rigid, for example, a vial, a pipet tip, a cell culture dish or array, or the base material may be substantially flexible along one or more planes, for example a fiber or membrane. From the viewpoint of maximizing the area of adsorption and/or immobilization and enhancing the efficiency of adsorption and/or immobilization, the use of fibrous materials is advantageous. Grafted fibers in such membrane compositions or sheets thereby provide a substantially enhanced brush surface area.

Woven fiber sizes appropriate for the present invention range from about 10 nm to about 100,000 nm. It is particularly advantageous to use woven fibrous materials having fiber diameters of from about 1000 nm to about 50,000 nm. One of the reasons why fibrous materials are advantageous is that they can be easily worked or woven into a desired shape, i.e., a fabric, and assembled in a device. Further, fibrous materials generally have no potential to release fine particles or dust into the atmosphere and, hence, they can be used in semiconductors and other areas of precision machining. If fibrous materials are to be used, they can be staple fibers or filaments. Such fibers can be processed into woven or nonwoven fabrics. If the membrane of the present invention employs a fibrous substrate, it can be used in admixture with other fibrous materials. Combinations of fibers thereby comprising different functional groups can be fabricated, thus providing for multifunctional properties in a single membrane composition.

Fibers can also be porous hollow fibers manufactured as nonwoven substrates. Examples of commercially available porous hollow fibers are those manufactured by Asahi Chemical Industry, Corp., described herein. These can have a broad range of porosity and be fabricated into, for example, filtration devices. Furthermore, a combination of porosity and fiber composition thereby provides physical and molecular immobilization, filtration or concentration. If fibrous materials are to be used in a spherical form, their diameter is advantageously adjusted to lie between about 2 and 20 mm, simply from the viewpoint of ease of handling. The porosity of the base material of the present invention has an average pore diameter of about 0.1 nm to about 50,000 nm, and preferably about 1 nm to 5000 nm, and more preferably 10 to 1000 nm from the standpoint of the desired functional activity and permeability of the base material. One skilled in the art could determine the optimal composition and porosity for a given application. When the average pore diameter is too small, the permeability of the membrane composition is decreased. When the average pore diameter is too large, desired substances would are not well adsorbed on the brush surface of the porous base material. Instead, the subject sample would pass through the pores of the porous base material without contacting the brush surface and functional groups, so that the activity of the desired functional group cannot be attained. The porosity of the porous base material of the present invention is preferably in the range of from 20 to 90%, more preferably 50 to 90%. The degree of porosity depends e.g., on the physical properties of the base material used. Measurement of porosity and pore size etc. of a base material is generally well known in the art, for example, the bubble point method, mercury pressure method, Scanning Electron Microscopy (SEM) or Tunneling Electron Microscopy (TEM) or the nitrogen adsorption method (see, ASTM F316, 1970; Pharmaceutical Tech., 1978, 2:65-75; Filtration in the Pharmaceutical Industry, Marcel Dekker, 1987, incorporated herein by reference).

An example of a rigid container of the present invention is described in detail as Example Three. In this Example, the base material is formed into a disposable plastic tip for a microvolume pipet device. The pipet tip comprises polymer brushes having one or more functional groups immobilized on the lumenal surface in multi-layers. A semi-rigid container, i.e., tubing, is described in Example Six. The tubing comprises polymer brushes having one or more functional groups immobilized on the lumenal surface in multi-layers. However, the invention is suitable for fabrication into powders, sheets, membranes or films, porous or non-porous materials, hollow fibers, woven fibers and fabrics, vials, containers and similar articles of manufacture.

Agents for Generating Radicals

The agent generating radicals which are capable of creating radical sites is an organic peroxide or a perester such as, for example, tert-butylperoxy 3,5,5-trimethylhexanoat-e, 2,5-dimethyl-2,5-di(benzoylperoxy)hexane, tert-butyl-peroxy 2-ethylhexyl carbonate, tert-butylperoxy acetate, tert-amylperoxy benzoate, tert-butylperoxy benzoate, 2,2-di(tert-butylperoxy)butane, n-butyl 4,4-di(tert-butyl-peroxy)valerate, ethyl 3,3-di(tert-butylperoxy)-butyrate, dicumyl peroxide, tert-butyl cumyl peroxide, di-tert-amyl peroxide, di(2-tert-butylperoxyisopropyl)benzene, 2,5-dimethyl-2,6-di(ter-t-butylperoxy)hexane, di-tert-butyl peroxide, 2,5-dimethyl-2,5-di-tert-but-ylperoxy-3-hexyne, 3,3,6,6,9,9-hexamethyl-1,2,4,5-tetraoxacyclononane, tert-butyl hydroperoxide, 3,4-dimethyl-3,4-diphenylhexane, 2,3-dimethyl-2,3-diphenylbutane and tert-butyl perbenzoate and azo compounds, for example azobisisobutyronitrile and dimethyl azodiisobutyrate; the said agent is preferably chosen within the group consisting of dicumyl peroxide, tert-butyl cumyl peroxide, di-tert-amyl peroxide, di-tert-butyl peroxide and 2,5-dimethyl-2,5-di(tertbutylperoxy)-3-hexyne.

Radiation Induced Graft Polymerization

Graft polymerization can be carried out, for example, by polymerization in the presence of a chemical or inducible polymerization initiator, thermal polymerization, irradiation-induced polymerization using ionizing radiation (e.g., alpha rays, beta rays, gamma rays, accelerated electron rays. X-rays, or ultraviolet rays). Polymerization induced by gamma rays or accelerated electron rays provides a convenient graft polymerization method.

Several methods of graft polymerization of a reactive monomer to a base material exist. The base material can be a formed article or can be manufactured into a product or device at a later time. Liquid phase polymerization, in which a formed article is directly reacted with a liquid reactive monomer, and gaseous or vapor phase polymerization, in which a formed article is brought into contact with vapor or gas of a reactive monomer, are two polymerization methods that are useful in the present invention according to the end use or purpose. Vapor phase grafting is described in J. Membr. Sci. 1993, 85:71-80, Chem. Mater. 1991, 3:987-989, Chem. Mater. 1990, 2:705-708, and AIChE J. 1996, 42:1095-1100, all of which are herein incorporated by reference.

Graft polymerization of the reactive monomer to the base material is performed. Grafting proceeds in three different ways: (a) pre-irradiation; (b) peroxidation and (c) mutual irradiation technique. In the pre-irradiation technique, the first polymer backbone is irradiated in vacuum or in the presence of an inert gas to form radicals. The irradiated polymer substrate is then treated with the monomer, which is either liquid or vapor or as a solution in a suitable solvent. However, in the peroxidation grafting method, the trunk polymer is subjected to high-energy radiation in the presence of air or oxygen. The result is the formation of hydroperoxides or diperoxides depending on the nature of the polymeric backbone and the irradiation conditions. The peroxy products, which are stable, are then treated with the monomer at higher temperature, whence the peroxides undergo decomposition to radicals, which then initiate grafting. The advantage of this technique is that the intermediate peroxy products can be stored for long periods before performing the grafting step. On the other hand, with the mutual irradiation technique the polymer and the monomers are irradiated simultaneously to form the radicals and thus addition takes place. Since the monomers are not exposed to radiation in the preirradiation technique, the obvious advantage of that method is that it is relatively free from the problem of homopolymer formation which occurs with the simultaneous technique. However, the decided disadvantage of the pre-irradiation technique is the scission of the base polymer due to its direct irradiation, which brings forth predominantly the formation of block copolymers rather than graft copolymers.

The base material substrate surfaces activated in this way are coated in a solution comprising reactive monomers, for example, tert-butylaminoethyl methacrylate, by known methods, such as by dipping, spraying or brushing. Suitable solvents have proved to be water and water/ethanol mixtures, although other solvents can also be used if they have a sufficient dissolving power for tert-butylaminoethyl methacrylate, and wet the base material substrate surfaces thoroughly. Solutions having reactive monomer contents of 0.1% to 10% by weight, for example about 5% by weight, have proved suitable in practice and in general give continuous coatings which cover the substrate surface and have coating thicknesses which can be more than 0.1 μm in one pass. Two, three, or more different reactive monomers can be cografted to the base material, see, Chem. Mater. 1999, 11:1986-1989, J. Membr. Sci. 1993, 81:295-305, J. Electrochem. Soc. 1995, 142:3659-3663, and React. Polym. 1993, 21:187-191, all incorporated herein by reference.

A reactive monomer is any compound that is capable of participating in a radical induced graft polymerization reaction. The reactive monomer thus incorporates in the side chain reaction, and forms polymer brushes. The term monomer is used for simplicity, as side reactions between reactive monomers can create oligomers before these are in turn involved in the polymerization reaction with the base material, and oligomers or even polymers are also useful reactive species for the present invention. As described above, monomer side chain brushes can be obtained, comprising multiple functional groups, i.e., three functional groups on a single monomeric brush.

The base material and reactive monomer may be the same compound, for example, a polyethylene base material may utilize ethylene monomers or polymers in the grafting reaction. Reactive monomers that can be used in the present invention include, for example, vinyl monomers and heterocyclic monomers. Other specific examples of suitable reactive monomers include vinyl monomers containing a glycidyl group, e.g., glycidyl methacrylate, glycidyl acrylate, glycidyl methylitaconate, ethyl glycidyl maleate, and glycidyl vinyl sulfonate; and vinyl monomers containing a cyano group, e.g., acrylonitrile, vinylidene cyanide, crotononitrile, methacrylonitrile, chloroacrylonitrile, 2-cyanoethyl methacrylate, and 2-cyanoethyl acrylate. These have epoxide groups for immobilization of functional groups and vinyl groups, which provide reactive polymerization sites and are thereby useful as reactive monomers. Ring-opening, i.e., the conversion of the epoxy groups into diol groups of the poly-GMA brushes is described in J. Membr. Sci. 1996, 117:33-38 (incorporated by reference).

The reactive monomers are covalently bonded to the base material through the polymerization reaction, or are separately formed and affixed or adhered to the base material. The reactive monomers form polymer brushes that are thereby grafted to the base material. The degree of grafting is determined by the choice of base material and reactive monomer, the polymerization method, and the desired length and width of the brushes. In certain cases, the resultant polymer brushes of the invention have bioactive properties themselves, for example, tert-butylaminoethyl methacrylate on a surface of an article or apparatus displays antimicrobial activity.

Measurement of modified or grafted materials can be determined by, for example degree of grafting, assaying thickness or weight, water content, IR method (FTIR-ATR, etc), titration for ion-exchange groups, zeta-potential, Donnan method, atomic force microscopy (AFM), scanning electron microscopy (SEM), determination of contact angle, XPS (X-ray photoelectron spectroscopy), and SIMS (secondary ion mass spectrometry).

The grafting copolymerization of the reactive monomer applied to the activated surfaces is also effected by radical induced polymerization initiated by, for example, short wavelength radiation in the visible range or in the long wavelength segment of the UV range of electromagnetic radiation. The radiation of a UV-Excimer of wavelengths 250 to 500 nm, preferably 290 to 320 nm, for example, is particularly suitable. Mercury vapor lamps are also suitable here if they emit considerable amounts of radiation in the ranges mentioned. The exposure times generally range from 10 seconds to 30 minutes, preferably 2 to 15 minutes. A suitable source of radiation is, for example, a UV-Excimer apparatus HERAEUS Noblelight, Hanau, Germany. However, mercury vapor lamps are also suitable for activation of the substrate if they emit considerable proportions of radiation in the ranges mentioned. The exposure time generally ranges from 0.1 second to 20 minutes, preferably 1 second to 10 minutes.

The activation of the reactive monomers and base materials with UV radiation can furthermore be carried out with an additional photosensitizer. Suitable such photosensitizers include, for example, benzophenone, as such are applied to the surface of the substrate and irradiated. In this context, irradiation can be conducted with a mercury vapor lamp using exposure times of 0.1 second to 20 minutes, preferably 1 second to 10 minutes.

According to the invention, the activation can also be achieved by a high frequency or microwave plasma (Hexagon, Technics Plasma, 85551 Kirchheim, Germany) in air or a nitrogen or argon atmosphere. The exposure times generally range from 30 seconds to 30 minutes, preferably 2 to 10 minutes. The energy output of laboratory apparatus is between 100 and 500 W, preferably between 200 and 300 W. For example, a Corona apparatus (SOFTAL, Hamburg, Germany) can furthermore be used for the polymer activation. In this case, the exposure times are, as a rule, 1 to 10 minutes, preferably 1 to 60 seconds.

The flaming of surfaces likewise leads to activation of the reactive monomers and base materials. Suitable apparatus, in particular those having a barrier flame front, can be constructed in a simple manner or obtained, for example, from ARCOTEC, 71297 Monsheim, Germany. The apparatus can employ hydrocarbons or hydrogen as the combustible gas. In all cases, harmful overheating of the base materials must be avoided, which is easily achieved by intimate contact with a cooled metal surface on the substrate surface facing away from the flaming side. Activation by flaming is accordingly limited to relatively thin, flat base materials. The exposure times generally range from 0.1 second to 1 minute, preferably 0.5 to 2 seconds. The flames without exception are nonluminous and the distances between the substrate surfaces and the outer flame front ranges from 0.2 to 5 cm, preferably 0.5 to 2 cm.

In the case of ionizing radiation initiated polymerization, in addition to the ultraviolet radiation discussed above, electron beams, X-rays, alpha rays, beta rays, gamma rays, etc., can be used. Graft polymerization condition changes with such variables, as the crystalline and amorphous structure of the base material polymer, the influence of solvent or gasses, temperature, pH, the hydrophobicity/hydrophilicity of the base material, reactive monomers, irradiation dose and intervals of exposure, and the type of radicals generated by irradiation. One skilled in the art would recognize such variables and adjust experimental conditions accordingly, for example activation by electron beams or gamma-rays, from a cobalt-60 source allow short exposure times which generally range from about 0.1 to about 60 seconds and employ dose ranges of about 1 to about 500 kGy. These high energy radiation sources are appropriate for applications where it is desirable to initiate a radical induced polymerization reaction on one or more intraluminal surfaces of a base material.

Multiple grafting steps can also be used to create the polymer brushes. Radicals are generated in the base material, for example a polymer base material is irradiated at an ambient temperature under nitrogen atmosphere to create radicals for polymer grafting. In the currently preferred embodiment, irradiation is performed by using an electron beam accelerator. Graft polymerization of reactive monomers (for example, liquid phase grafting) is performed on the base material to allow the formation of polymer brushes. As such, grafted polymer #1 is obtained. The above processes are repeated to obtain grafted polymer #2, grafted polymer #3 and so on. Moreover, the grafting process can be stopped at any step depending on the desired complexity of the brush structure. Different reactive monomers can be used at each grafting step, providing a plurality of brush compositions for immobilizing numerous types of functional groups or bioactive molecules thereto. The process can include immobilization of functional groups followed by additional grafting reactions.

Functional Brushes

The present invention provides compositions and methods for radical induced polymerization of base materials or grafting of polymer brushes formed by radical induced polymerization to the base materials, thereby providing base materials having a plurality of polymer brush structures. These polymer brush structures have physical properties themselves, due to, for example, their size, brush density and brush morphology. However the invention further provides that the polymer brushes have functional groups immobilized thereto. Methods of immobilizing functional groups to a substrate are well known, and are appropriate for immobilizing functional groups to the brushes (see, J. Membr. Sci. 1993, 76:209-218, incorporated herein by reference). One or more types of functional groups can be immobilized to the brushes, i.e., one, two, three, four, or five or more different types of functional groups, depending on the desired functionality.

Agents for Binding Functional Groups to the Brushes

While the base material itself is generally a material that is essentially nonreactive, or inert, the invention permits the use of a reactive base material. In contrast, the polymer brushes comprise one or more reactive groups on the brush surface, permitting functional or multifunctional polymer brushes. The base material and polymer brushes respectively can therefore assume two different functional parts of the invention.

Different methods for immobilization of functional groups include, for example, physical adsorption (non-covalent bridges such as ionic and hydrogen bonds, hydrophobic interactions and van der Waals forces), immobilization via reactive groups, aminopropyltriethoxysilane bridges, glutaraldehyde, or bis(sulfosuccinimidyl) suberate activation, or via aldehydye groups, phosphoramidite groups, peptide groups, binding through biotin or avidin, protein A or G, attachment via metal-carrying media, such as chelate-forming iminodiacetate groups, copper ions, nickel ions, ferric or ferrous ions, zinc ions, magnesium ions, manganese ions, cobalt ions or similar charged species including complexes of the same, covalent attachment of oxidized groups, for example to oxidize the carbohydrate moieties in an antibody's Fc region with periodate to form aldehyde groups, which are then chemically bound to hydrazide-activated solid supports such as agarose, silica, acrylic-based copolymers, and cellulose. Methods for immobilization of nucleic acids include, for example, adsorption: (i) electrochemical adsorption: electrostatic attraction between the positively charged solid support and the negatively charged oligonucleotides. (ii) hybridization between electrochemically adsorbed oligonucleotides and its complementary target for sequence specific hybridization, avidin-biotin complexation, covalent attachment: (i) through deoxyguaosine group using carbodiimide method (in other words, carboxylic group (—COOH)), (ii) amino groups (—NH2), phosphoric acid groups. Organic synthesis (or peptide synthesis) can be performed directly on the polymer brushes or on functional groups immobilized thereto (see, U.S. Pat. No. 6,306,975, incorporated by reference). Other coupling chemistries are well known in the art, and by using graft polymerization, one can prepare solid supports having a plurality of functional groups (see, J. Biochem. Biophys. Methods 2001, 49:467480, Radiat. Phys. Chem. 1987, 30:263-270, Biosens. Bioelectron. 2000, 15:291-303, Analytica Chimica Acta 1997, 346:259-275, Chem. Rev. 2000, 100: 2091-2157, Tetrahedron 1998, 54: 15383-15443, Radiat. Phys. Chem. 1986, 27:265-273, and Solid-Phase Synthesis and Combinatorial Technologies by Pierfausto Seneci, John Wiley & Sons, Inc., 2000, all incorporated herein by reference).

Another method of immobilizing a molecule to the brush surface includes, without limitation, silanes of the formula SiX3-R, wherein X is a methyl group or a halogen atom such as chlorine and R is a functional group which can be a coating material as described herein or a group which is reactive with a coating material. Particular silane-terminated compounds include vinyl silanes, silane-terminated acrylics, silane-terminated polyethylene glycols (PEGs), silane-terminated isocyanates and silane-terminated alcohols. The silanes can be reacted with the surface by various means known to those skilled in the art. For example, dichloro methyl vinyl silane can be reacted with the surface in aqueous ethanol. This strongly binds to the surface via —O—Si bonds or directly with the silicon atom. The vinyl group of the silane can then be reacted with polymeric materials as described herein using appropriate conventional chemistries. For example, a methacrylate-terminated PEG can be reacted with the vinyl group of the silane, resulting in a PEG that is covalently bonded to the surface of the present device.

In addition, spacer molecules may be inserted between the functional group and the polymer brush, as is known in the art, to facilitate binding or improve the activity of the functional group or bioactive molecule. The extended morphology of the brushes can function as spacers, or additional chemical spacers can be used.

These functional groups impart to the compositions of the invention particular properties. For example, the functional groups can change the effective or active surface area and thereby change the adsorptive capacity. In certain embodiments, they provide for particular brush shapes. In other embodiments they impart a particular strength, chemical resistance, enzymatic property, affinity for a bioactive molecule, or other functional group or provide other effective functionality to the composition. Conventional ion-exchange resins do not rely upon the base material and functional groups to perform different functions.

Functional groups that are appropriate for immobilization by the brushes in the compositions of the present invention include, for example, ion exchange functional groups, i.e., anionically dissociating groups and cationically dissociating groups, hydrophilic functional groups, and other functional groups that have the ability to adsorb and/or immobilize other molecules.

One or more kinds of anionically dissociating substances can be immobilized by the polymer brushes. Examples of suitable anionically dissociating groups include quaternary ammonium salts and primary, secondary, and tertiary amino or amido groups. Specific examples include an amino group, a methylamino group, a dimethylamino group, and a diethylamino group. Preferred anionically dissociating groups include the amino group and quaternary ammonium salts. Reactive monomers that have such anionically dissociating groups and that are useful in the present invention include, for example, vinylbenzyltrimethyl ammonium salt, diethylaminoethyl methacrylate, dimethylaminoethyl acrylate, dimethylaminoethyl methacrylate, diethylaminoethyl acrylate, diethylaminomethyl methacrylate, tertiary-butylaminoethyl acrylate, tertiary-butylaminoethyl methacrylate and dimethylaminopropylacrylamide. Also useful in the present invention are reactive monomers that have epoxide groups capable of conversion to anionically dissociating groups. An example of such a reactive monomer is glycidyl methacrylate. An example of an amine capable of converting the epoxide group to an anionically dissociating group is diethylamine.

One or more kinds of cationically dissociating groups can be immobilized by the polymer brushes. Examples of such cationically dissociating groups include, for example, a carboxyl group, a sulfone group, a phosphate group, a sulfoethyl group, a phosphomethyl group, a carbomethyl group. Preferred cationically dissociating groups include a sulfone group and a carboxyl group. Reactive monomers that have such cationically dissociating groups and that are useful include, for example, acrylic acid, methacrylic acid, styrenesulfonic acid and salts thereof, and 2-acrylamido-2-methylpropanesulfonic acid.

One or more kinds of hydrophilic substances can be immobilized by the polymer brushes. Such hydrophilic groups are capable of trapping the water molecules present in air, forming a layer of adsorbed water on the surface of the membrane of the present invention. Such hydrophilic groups will function in water in the same manner as in air. Examples of suitable hydrophilic groups include, for example, a hydroxyl group, a hydroxyalkyl group (where the alkyl group is preferably a lower alkyl group), an amino group and a pyrrolidonyl group. Preferred hydrophilic groups include a hydroxyl group, a hydroxyalkyl group and a pyrrolidonyl group. One or more kinds of hydrophilic groups can be immobilized onto the polymer brush. Reactive monomers that have such hydrophilic groups and that are useful in the present invention include, for example, ethanolamine, hydroxyethyl methacrylate, hydroxypropyl acrylate, vinylpyrrolidone, dimethylacrylamide, ethylene glycol monomethacrylate, ethylene glycol monoacrylate, ethylene glycol dimethacrylate, ethylene glycol diacrylate, triethylene glycol diacrylate and triethylene glycol methacrylate. Thus a polymer brush may itself comprise a functional group, or one may be immobilized to the brush.

One or more kinds of functional groups can be immobilized on the polymer brushes. Such groups can be combined or immobilized in discrete multi-layers to impart an additional degree of functionality to the composition. For example, the present invention provides membrane compositions having enzymatic activity such as the ability to phosphorylate a polypeptide substrate, the ability to digest, i.e., a nucleic acid at a restriction site, or polypeptide, the ability to radiolabel a polynucleotide or polypeptide, or the ability to catalyze a biological or chemical reaction. Examples of enzyme functional groups that can be bound to or isolated using the polymer brushes, and potential uses for those enzymes, include, but are not limited to ascorbic acid oxidase (e.g., for avoidance of interference of ascorbic acid on diagnostic assays of blood, urine, or other samples), aspartase (e.g., for conversion of fumaric acid to L-aspartic acid), aminoacylase (e.g., for conversion of acetyl-D,L-amino acids to L-amino acids), tyrosinase (e.g., for synthesis of tyrosine from phenol, pyruvate and ammonia), lipase (e.g., for hydrolysis of a cyano-ester to ibuprofen or hydrolysis of a diltiazem precursor), penicillin amidase (e.g., for production of ampicillin and amoxycillin), hydantoinase and carbamylase (e.g., for hydrolysis of 5-p-HP-hydantoine to d-p-HP-glycine), DNase (e.g., for hydrolysis of DNA to oligonucleotides), bovine liver catalase (e.g., for hydrolysis of hydrogen peroxide), trypsin and chymotrypsin (e.g., for hydrolysis of whey proteins), arginase and asparaginase (e.g., for hydrolysis of arginine and asparagine), proteases (e.g., to remove organic stains from fabrics), lipases (e.g., to remove greasy stains from fabrics), amylase (e.g., to remove residues of starchy foods from fabrics), cellulase (e.g., to restore a smooth surface to fibers of fabrics and restore fabrics to their original colors), proteases and lipases (e.g., to intensify flavor and accelerate the aging process of foods), lactase (e.g., to produce low-lactose milk and related products for special dietary requirements), beta-glucanase (e.g., to help the clarification process of wines), cellulase (e.g., to aid the breakdown of cell walls in winemaking), cellulase and pectinase (e.g., to improve clarification and storage stability of wine), pectinase (e.g., to improve fruit-juice extraction and reduce juice viscosity), cellulase (e.g., to improve juice yield and color of fruit juice), lipase (e.g., for hydrolysis of fats and oils or the production of fatty acids, glycerine, fatty acids (e.g., used to produce pharmaceuticals, flavors, fragrances and cosmetics), alpha-amylase (e.g., for liquefaction of starch or fragmentation of gelatinized starch), aminoglucosidase (e.g., for saccharification or complete degradation of starch and dextrins into glucose), alpha-amylase (e.g., for conversion of starch to fructose), glucoamylase and pullulanase (e.g., for saccharification), glucose isomerase (e.g., for isomerization of glucose), beta-glucanase (e.g., for reduction of beta-glucans), beta-glucanase (e.g., for reduction of beta-glucans and pentosans), lipase, amidase and nitrilase (e.g., for manufacture of enantiomeric intermediates for drugs and agrochemicals), lipase (e.g., to remove fats in the de-greasing process in the leather industry), amylase and cellulase (e.g., to produce fibers from less valuable raw materials in the textiles industry), xylanase (e.g., as a bleaching catalyst during pretreatment for the manufacture of bleached pulp for paper), beta-galactosidase (e.g., for hydrolysis of lactose to glucose), trypsin and chymotrypsin (e.g., for hydrolysis of high-molecular-weight protein in milk), alpha-galactosidase and invertase (e.g., for hydrolysis of raffinose), alpha-amylase, beta-amylase, and pullulanase (e.g., for hydrolysis of starch to maltose), pectinase (e.g., for hydrolysis of pectins), endopeptidase (e.g., for hydrolysis of k-casein), protease and papain (e.g., for hydrolysis of collagen and muscle proteins), glucose oxidase and catalase (e.g., for conversion of glucose to gluconic acid), lipase (e.g., for hydrolysis of triglycerides to fatty acids and glycerol, hydrolysis of olive oil triglycerides, hydrolysis of soybean oil, butter oil glycerides and milk fat), cellulase and beta-glucosidase (e.g., for hydrolysis of cellulose to cellobiose and glucose), and fumarase (e.g., for hydrolysis of fumaric acid to 1-malic acid). Alternatively, microorganisms or fragments thereof can be functional groups, for example, such as Pseudomonas dacunhae (e.g., for conversion of L-aspartic acid to L-alanine), Curvularia lunata/Candida simplex (e.g., for conversion of cortexolone to hydrocortisone and prednisolone), or yeast (e.g., for fermentation of sugars and anaerobic fermentation); all can be immobilized on the polymer brushes.

The functional groups can include all hydrophilic groups, anionically dissociating groups and/or cationically dissociating groups, and enzymes. Stated more specifically, the polymer brush can include multiple functional groups (e.g., anionically dissociating groups and hydrophilic groups, or alternatively cationically dissociating groups and hydrophilic groups) or three kinds of functional groups (e.g., hydrophilic groups, anionically dissociating groups, and cationically dissociating groups), or more (e.g., hydrophilic groups, anionically dissociating groups, cationically dissociating groups, enzymes, SpA and one or more Fv antibody fragments). Combinations of functional groups that are appropriate in the present invention include, for example, an ionic group and a non-ionic group, i.e., an amine group with a coexisting hydrophilic group. A preferred embodiment additionally comprises a second functional group in combination with the first functional groups described above. In a currently preferred embodiment, the first, second, third, and fourth functional groups are immobilized on the polymer brushes in multilayers. Thus, one of the major features of the present invention is that different kinds of molecules having hydrophilic domains (non-ions) present in a sample solution with molecules having ionic domains (anions and/or cations), or molecules having a phosphorylation state, or a binding site or nucleotide or polypeptide sequence can be recovered, purified, concentrated and isolated, modified, synthesized, or otherwise utilized with the compositions of the invention. The functional group may be altered to change the binding of a substrate bioactive molecule, to thereby tailor the dissociation rate in vivo, and provide controlled release of the substrate bioactive molecule bound thereto. Such alteration or chemical modification may be effectuated on the compositions of the present invention, or the modifications may be effectuated before immobilization to the polymer brush surface.

The functional groups can include antibodies or domains or fragments thereof. Hydroxysuccinimide esters, for example, provide one method for immobilizing an antibody to the present composition via lysine residues. The carbohydrate moieties, described above, provide yet another source for immobilization to the polymer brushes or to functional groups. The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989). All such domains or fragments or sequences therefrom may be immobilized on polymer brushes by the methods described herein.

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv) but a bispecific antibody can be immobilized as described, and provides an additional functional property for the polymer brushes, i.e., an additional specificity for a ligand. Multiple isotypes, species, and epitope recognition properties can be imported to the polymer brushes by the methods described herein.

Humanized, or chimeric antibodies are also appropriate. Such approaches for generating these are further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430, 938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0 463 151 B1, grant published Jun. 12, 19961 International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety. Humanized, or chimeric antibodies or domains or fragments thereof can be immobilized to the polymer brushes as described.

Functionalized liposomes, microsponges and microspheres may also be immobilized to the materials described herein. Liposomes are lipid molecules formed into a typically spherically shaped arrangement defining aqueous and membranal inner compartments. Liposomes can be used to encapsulate agents within the inner compartments, and deliver such agents to desired sites within a cell. The agents contained by the liposome may be released by the liposome and incorporated into a cell, as for example, by virtue of the similarity of the liposome to the lipid bilayer that makes up the cell membrane. A variety of suitable liposomes may be used, including those available from NeXstar Pharmaceuticals or Liposome, Inc, if functionalized as by the procedures described herein. Liposomes may be immobilized to the polymer brushes by several methods, for example through interactions with the hydrophobic polymer brushes, or by a functional group, for example, a fatty acid functional group.

Microsponges are high surface area polymeric spheres having a network of cavities which may contain bioactive molecules. The microsponges are typically synthesized by aqueous suspension polymerization using vinyl and acrylic monomers. The monomers may be mono or difunctional, so that the polymerized spheres may be cross-linked, thus providing shape stability. Process conditions and monomer selection can be varied to tailor properties such as pore volume and solvent swellability, and the microsponges may be synthesized in a controlled range of mean diameters, including small diameters of about 2 micrometers or less. A standard bead composition would be a copolymer of styrene and di-vinyl benzene (DVB). The agents contained by the polymeric microsponges may be gradually released therefrom due to mechanical or thermal stress or sonication. A variety of suitable microsponges may be used, if functionalized as by the procedures described herein, including those commercially available from Advanced Polymer Systems. These can be grafted to the polymer brushes or otherwise immobilized by standard chemical techniques known in the art in view of the teachings described herein.

Thus, the resulting base material comprises a plurality of polymer brushes which further comprise one or more functional groups immobilized thereto. These compositions provide a wide range of combinations, and are useful in diverse processes, for example, the products and processes disclosed herein, as well as similar applications known to those of skill in the environmental, filtration, medical, pharmaceutical and biotechnology arts. Such equivalent compositions and processes are considered to be within the scope of the invention.

The invention will be further described in the following examples, which do not limit the scope of the invention described in the claims.

### EXAMPLE ONE

### Preparation of Membrane Compositions for the Immobilization of Ascorbic Acid Oxidase

A base material comprising a porous membrane in a hollow-fiber form was used as a trunk polymer for grafting. This hollow fiber, made of polyethylene, had inner and outer diameters of 1.8 and 3.1 mm, respectively, with an average pore size of 0.4 microns and porosity of 70%. The reactive monomer, glycidyl methacrylate (GMA, CH₂═CCH₃COOCH₂CHOCH₂) was purchased from Tokyo Kasei Co., Ltd., and used without further purification. A preparation scheme of porous hollow-fiber membrane compositions containing a diethylamino (DEA) group as an anion-exchange group consists of four steps. (1) Irradiation of an electron beam onto the trunk polymer to form radicals: the polyethylene porous hollow-fiber membrane was irradiated by an electron beam in a nitrogen atmosphere at ambient temperature using a cascade-type accelerator (Dynamitron model IEA 3000-25-2, Radiation Dynamics Inc., New York). The dose was set at 200 kGy. (2) Grafting of a reactive monomer: the irradiated base material membrane was immersed in 10 v/v % GMA/methanol solution at 313 K for 12 min (J. Membr. Sci, 71:1-12, 1992, incorporated by reference). (3) Introduction of an anion-exchange functional group for selective binding of a target protein: the GMA-grafted membrane was reacted with 50 v/v % diethylamine (DEA)/water solution at 303 K for 2 h. (4) Blocking of nonselective adsorption of other proteins: the unreacted epoxy groups were converted into an inert 2-hydroxyethylamino group by the immersion of the membrane in ethanolamine (EA) at 303 K for 6 h. The resultant composition is a porous hollow-fiber membrane that is referred to as a DEA-EA fiber.

Immobilization of Ascorbic Acid Oxidase onto the Membrane Compositions

Ascorbic acid oxidase was supplied by Asahi Chemical Industry Co., Ltd., Japan. Other chemicals were of analytical grade. In order to immobilize ascorbic acid oxidase (AsOM) as an enzyme functional group onto the DEA-EA fiber, the following solution was subsequently permeated through the pores of the 2-cm-long DEA-EA fiber using a syringe pump at a constant permeation rate of 1 ml/min at ambient temperature: (1) 14 mM Tris-HCl buffer (pH 8.0) for equilibration, (2) 0.50 g of the enzyme per L of the buffer to bind the enzyme to the diethylamino-group-containing polymer chains grafted onto the pores of the fiber, (3) the buffer to wash the pores, (4) 0.50 wt % glutaraldehyde aqueous solution to cross-link the enzymes immobilized by the polymer brushes, and (5) 0.50 M NaCl to elute the uncrosslinked enzyme. Through a series of the above procedures, the enzyme concentration in the effluent penetrating the outside surface of the hollow fiber was determined by measuring UV absorbance at 235 nm. The amount of the enzyme immobilized via ion-exchange adsorption and subsequent crosslinking, Q, was calculated as follows:
Q(mg/g)=[(amount adsorbed)−(amount washed)−(amount uncrosslinked)]/(mass of membrane in a dry state)
The resultant porous hollow-fiber membrane immobilizing the ascorbic acid oxidase is referred to as an AsOM fiber.

Activity Determination During Permeation Through The Membrane Compositions The 2-cm-long AsOM fiber was set in an I-configuration. For conditioning of the AsOM fiber, 20 mM acetate buffer (pH 4.0) was forced to permeate outward across the AsOM fiber at a constant permeation rate of 30 ml/h. Then, ascorbic acid (AsA) solution as a substrate solution, the AsA concentration of which ranged from 0.025 to 0.10 mM, was fed from the inside surface of the AsOM fiber to the outside, where the permeation rate ranged from 30 to 150 mL/h. Space velocity (SV) was defined as:
SV(h⁻¹)=(permeation rate of the AsA solution)/(AsOM fiber volume including the lumen part)

The concentration of ascorbic acid in the effluent was continuously determined by measuring UV absorbance at 245 nm. The conversion of AsA to dehydroascorbic acid and the activity were defined as:
Conversion (%)=100 [(1−(AsA conc. in the effluent)/(AsA conc. in the feed))]
Activity (mol/h/L)=(SV) [(AsA conc. in the feed)−(AsA conc. in the effluent)]

In order to examine the storage stability of the AsOM fiber, a similar experiment was performed on another AsOM fiber after a storage period of up to 25 days at 283 K in the buffer solution.

### EXAMPLE TWO

### Preparation of Membrane Compositions for the Immobilization of Aminoacylase

A commercially available porous hollow-fiber membrane, supplied by Asahi Chemical Industry Co. (Tokyo, Japan), was used as a trunk polymer for grafting. This hollow fiber had inner and outer diameters of 1.2 and 2.2 mm, respectively, with an average pore diameter of 0.24 microns and a porosity of 70%. Aminoacylase was purchased from Sigma Co. (No.3333). Glycidyl methacrylate (CH₂═CCH₃COOCH₂CHOCH₂) was obtained from Tokyo Chemical Co., and was used without further purification. Other chemicals were of analytical grade or higher. An anion-exchange porous membrane with a hollow-fiber form was prepared by radiation-induced graft polymerization and subsequent chemical modifications (J. Chromatogr. A., 689:211-218, 1995, incorporated by reference). The trunk polymer was irradiated with an electron beam at a dose of 200 kGy and immersed in 10 (v/v)% glycidyl methacrylate(GMA)/methanol solution at 313 K for 12 minutes. The degree of GMA grafting, defined below, was 160%. The GMA-grafted hollow fiber was immersed in 50 (v/v)% aqueous solution of diethylamine (DEA) at 303 K for 1 h and subsequently in ethanolamine (EA) at 303 K for 6 h. The molar conversion of epoxy groups into anion-exchange groups was calculated from the weight gain. The resultant hollow fiber was referred to as a DEA-EA fiber.

Conditioning of Anion-Exchange Porous Membrane Compositions

Before the adsorption of aminoacylase to the DEA-EA fiber in a permeation mode, the DEA-EA fiber was conditioned by being immersed in either 1M HCl or 1M NaOH at 303 K for 1 h and then thoroughly rinsed with ultrapure water. The resultant fibers with HCl and NaOH are referred to as DEA/Cl and DEA/OH fibers, respectively. For comparison, the DEA-EA fiber, i.e., the unconditioned fiber, was used for enzyme binding. The swelling ratio is defined as the volume ratio in the wet state of the conditioned fiber to the unconditioned fiber. Subsequently, the swelling ratio was determined after the immersion of the fiber in 0.5 M NaCl and subsequent washing with ultrapure water.

Immobilization of Aminoacylase onto the Hollow Fiber

A 7-cm-long or 2-cm-long DEA-EA fiber was positioned in a I-shaped configuration. Aminoacylase was dissolved in 14 mM Tris-HCl buffer (pH 8.0) to a concentration of 1.0 mg/ml. Aminoacylase solution was fed to the inside surface of the DEA-EA fiber. The solution was allowed to permeate through the pores across the membrane thickness at a constant flow rate of 60 ml/h. The effluent penetrating the outside surface of the hollow fiber was continuously sampled. Aminoacylase in the effluent was determined by measuring the UV absorbance at 280 nm. Experiments were performed at ambient temperature. The amount of the enzyme adsorbed was evaluated by the following integration:
Q=∫₀^{Ve}(C₀C)dV/W
where C₀ and C are the enzyme concentrations of the feed and effluent, respectively. The terms V, Vₑ, and W are the effluent volume, the effluent volume where C reaches C₀, and the weight of the hollow fiber, respectively. Subsequently, the aminoacylase-adsorbed hollow fiber was immersed in 0.05 wt % glutaraldehyde solution (pH 8.0) for 17 h at 303 K to cross-link the enzymes captured by the side chains. The uncross-linked enzyme was eluted by permeating 0.5 M NaCl through the pores, and its concentration was determined. The amount of aminoacylase immobilized after cross-linking was evaluated by
   Amount of aminoacylase immobilized (mg/g)=[(amount adsorbed)−(amount eluted)]/(weight of hollow fiber)
   Percentage of cross-linking (%)=100 (amount immobilized)/(amount adsorbed)
   The resultant hollow fiber was referred to as an aminoacylase-immobilized fiber.

Determination of the Activity of Aminoacylase-Immobilized Membrane Compositions

Acetyl-DL-methionine (Ac-DL-Met) was selected as a substrate for aminoacylase. The aminoacylase-immobilized fiber was positioned in an I-shaped configuration. The Ac-DL-Met solution was allowed to permeate through the pores of the aminoacylase-immobilized fiber using a syringe pump (ATOM, 1235N) at a flow rate ranging from 30 to 180 ml/h; the space velocity, defined above, varied from 40 to 200 h⁻¹. The effluent was sampled to determine the concentration of L-Met according to the ninhydrin method (Biotechnol. Bioeng, 19:311-321, 1977, incorporated by reference). The conversion of the Ac-DL-Met into L-Met and the activity of the fiber were defined as:
Conversion (%)=100 (moles of L-Met produced)/(moles of DL-Met fed)
Activity (mol/L/h)=[(conversion)/100] (feed concentration) (SV).

### EXAMPLE THREE

### Functionalized Polymeric Tools

Grafting of poly-GMA Brushes onto Plastic Pipet Tips

A container of the present invention includes a functionalized pipet tip. Commercially available pipette tips were purchased from Eppendorf-Netheler-Hinz GmbH (Standartips 300 μL). The pipette tips were made of polypropylene. Pipette tips were set in a polyethylene package which was subsequently sealed with N₂. Electron beam irradiation was performed at ambient temperature by means of a cascade electron accelerator (Dynamitron IEA-3000-25-2, Radiation Dynamics, Inc.) operated at a voltage of 2 MeV and a current of 1 mA. The conveyer on which the polyethylene fibers were mounted was reciprocated at a speed of 3.8 cm/s. The irradiation dose per passage of the conveyer was 10 kGy. The exposed total irradiation dose of electron beam was set at 50, 100, 150, or 200 kGy. After irradiation the fibers were immersed in a GMA solution (10 vol/vol % in methanol or butanol) previously deaerated by nitrogen bubbles and reacted at 313 K under vacuum for a predetermined time. After the grafting of GMA, the fibers were washed with dimethylformamide and methanol, and then dried under reduced pressure. The amount of GMA graft polymerized is defined as:
Degree of grafting=[(W₁−W₀)/W₀]*100%
Density of polymer brush [mol/m²]=(W₁−W₀)/1421A
where W₀ and W₁ are the weights of the original and GMA-grafted pipette tips, respectively, and A is the total surface area of the pipette tip. The constant 142 is the molecular mass of GMA. The epoxy group of the poly-GMA chains appended onto the surface of the pipette tip was converted into cation- and anion-exchange groups by reaction with sodium sulfite and trimethylamine, respectively. The density of the immobilized ion-exchange groups was evaluated from the weight gain as follows:
Ion-exchange group density [mol/m²]=(W₂−W₁)/Mr/A
where Mr is the molecular mass of the reagent for modification. The remaining epoxy groups were converted into diol groups, or 2-hydroxyethylamino and trimethylamino groups for the preparation of cation- and anion-exchange pipette tips, respectively.

Functionalization of Poly-GMA Brushes with Sulfonic Acid Groups and Trimethylamine Groups

The epoxy groups on poly-GMA brushes were converted into sulfonic acid (SO₃H) groups by immersing the GMA-grafted pipette tips in a sulfonating reagent (Sodium hydrogensulfite (SS) solution comprising SS/isopropyl alcohol (IPA)/water:10/15/75 in weight ratio. After sulfonation, the remaining epoxy groups were hydrophilized with sulfuric acid. The epoxy groups of the poly-GMA brushes were reacted with diethylamine (DEA 50 vol/vol % in water) or trimethylamine-HCL (TMA-HCl/IPA/Water=Oct. 15, 1975 in weight %). After the introduction of the quaternary ammonium salt groups, the remaining epoxy groups were hydrophilized with ethanolamine. The surface of the pipet tip in a dry state was observed by scanning electron microscopy (SEM). The performance of these grafted pipet tips are summarized in Tables C, D and E, and discussed below.

TABLE C
Adsorption of proteins by ion-exchange pipette tips

| | | Solution | | Number of |
|---|---|---|---|---|
| | Pipetting | volume | Pipetting | pipetting |
| Steps | solution | [μL] | volume | times |
| 1. Conditioning | Buffer* | >500 | 200 | 30 |
| 2. Adsorption | 0.5 g/L protein | 200 | 150 | Specified |
| | solution in | | | number |
| | buffer* | | | of times |
| 3. Washing | Buffer | 200 | 170 | 30 |
| 4. Elution | 0.5 M NaCl in | 200 | 170 | 80 |
| | buffer | | | |

*For SS-Diol tips: lysozyme as protein, carbonate buffer (pH 9.0). For DEA-EA or TMA tips: bovine serum albumin (BSA) as protein, Tris-HCl buffer (pH 8.0).

TABLE D
Adsorption of lysozyme by cation-exchange (ss-diol) pipette tip

| Base tip | White | Platinum | Yellow | Yellow | Long |
|---|---|---|---|---|---|
| Solvent | Methanol | Methanol | Methanol | 1-Buthanol | Methanol |
| Total irradiation dose [kGy] | 50 | 50 | 200 | 200 | 200 |
| Monomer concentration [vol %] | 50 | 50 | 10 | 10 | 10 |
| Degree of grafting [%] | 12 | 12 | 5.3 | 4.3 | 4.8 |
| Grafted amount per surface area [g/m²] | 30 | 30 | 15 | 11 | |
| Conversion [%]: | | | | | |
| Weight method | 64 | 64 | 85 | 88 | 90 |
| Titration method | 37 | 32 | 72 | 61 | — |
| Ion exchange group concentration [mmol]: | | | | | |
| Weight method | 0.17 | 0.17 | 0.15 | 0.11 | |
| Titration method | 0.097 | 0.078 | 0.095 | 0.066 | |
| Adsorption of lysozyme by pipetting: | | | | | |
| Adsorption amount | 7.3 | 7.0 | 62 | 50 | 77 |
| (128 s residence time) [μg] | | | | | (140 s) |
| Elution ratio [%] | — | — | 100 | 100 | |

TABLE E
Adsorption of BSA by anion-exchange pipette tip

| Base tip | Yellow | Yellow | Yellow | Long |
|---|---|---|---|---|
| Solvent | Methanol | 1-Buthanol | IPA/water | IPA/water |
| Total irradiation dose [kGy] | 200 | 200 | 200 | 200 |
| Monomer concentration [vol %] | 10 | 10 | 10 | 10 |
| Degree of grafting [%] | 4.9 | 4.7 | 7.2 | 7.4 |
| Grafted amount per surface area [g/m²] | 14 | 13 | | |
| Functionalization reagent | Diethylamine | Diethylamine | Trimethylamine-HCl | Trimethylamine-HCl |
| Reagent concentration | 50 vol % in | 50 vol % in | Monomer/IPA/water = | Monomer/IPA/water = |
| | water | water | 10/15/75 in weight % | 10/15/75 in weight % |
| Conversion [%]: | | | | |
| Weight method | 100 | 88 | 46 | 26 |
| Ion exchange group concentration [mmol]: | | | | |
| Weight method | 0.10 | 0.086 | | |
| Adsorption of BSA by pipetting: | | | | |
| Adsorption amount | 7.2 | 6.9 | 7.5 | 20 |
| (128 s residence time) [μg] | | | | (140 s) |
| Elution ratio [%] | 100 | 100 | | |

Protein Collection with Ion-Exchange Tips

Hen egg lysozyme (HEL), a positively charged protein, in a solution of 0.5 mg/mL buffered with carbonate buffer (pH 9.0), and BSA, a negatively charged protein, in a solution of 0.5 mg/mL buffered with Tris-HCl buffer (pH 8.0) were used to evaluate the protein collection performance of the cation and anion exchange tips. 150 μL of protein solution at ambient temperature—about 22° C.—was introduced into the ion-exchange pipette tips, held in the tip for 1.4 seconds, and then discharged to a fresh sample vial. This stepwise process of aspiration and discharge is referred to as a cycle. The protein concentration in the vial was determined by the Bradford method (BIORAD, Protein assay kit).

For comparison, commercially available ion-exchange pipette tips, POROS-Tip HS and HQ, were purchased from PE Biosystems, and their performance was evaluated according to the same procedures as described above. The manual pipette (GILSON, Pipetman 200) was employed for the bead-packed pipette tips due to their higher pressure loss than the SS and TMA tips. These tips are described in U.S. Pat. No. 6,048,457 and U.S. Pat. No. 6,200,474, each incorporated by reference in their entirety.

Introduction of the ion-exchange group into the polymer brush increased the roughness of the lumenal surface of the pipette tip. This demonstrates that electrostatic repulsion of the ion-exchange or charged groups of the polymer brush induced the expansion of the polymer brush.

Unlike the conventional pipette tips that were packed with ion-exchange beads, the pipette tips were immobilized with ion-exchange polymer brushes directly on their surface by radiation-induced graft polymerization and subsequent chemical modification. In comparison with the commercially available ion-exchange pipette tips, at the top of which the ion-exchange beads were packed, the lower pressure loss for the flow-through of the protein solution was demonstrated. Decrease of HEL and BSA concentrations for sample solutions cycled through the grafted cation- and anion-exchange pipette tips, respectively, was ascertained as described.

### EXAMPLE FOUR

### Order Variation of Successive Modifications of Polymer Brushes Governs the Degrees of Their Expansion and Protein Multi-Layering

Poly-glycidyl methacrylate brushes were appended onto a porous hollow-fiber membrane with a pore size of 0.4 μm and a porosity of 70% by radiation-induced graft polymerization. Diethylamino or sulfonic acid functional groups as an ionizable functional group and 2-hydroxyethylamino or diol functional groups as a coexisting group were immobilized onto the polymer chains. The variation in the order of successive chemical modifications of the introduction of the ionizable functional group and the coexisting hydrophilic functional group determined the degree of the extension of the polymer brushes grafted onto the pore surface. The liquid permeability and protein adsorptivity of the resultant four kinds of porous hollow-fiber membranes immobilizing the ionizable polymer brushes were determined in a permeation mode to quantitatively evaluate the degree of the expansion of the polymer brushes. The polymer brushes modified with the ionizable functional group at the first step exhibited the higher degrees of their expansion and protein multilayer binding at the lower conversion of the epoxy group into the ionizable functional group. To observe the identical degree of multilayer binding of hen-egg lysozyme by the polymer brushes immobilizing the sulfonic acid and diol functional groups, conversions of 10 and 60% of the epoxy group into the sulfonic acid group for the polymer brushes sulfonated at the first step and that at the second step, respectively.

The order of successive chemical modifications after the graft polymerization of an epoxy-group-containing reactive monomer, i.e., introduction of ionizable and coexisting hydrophilic functional groups, can govern the degree of the expansion of the polymer brushes because the ionizable moiety density is variable along the polymer brushes. Therefore, determination of water permeability and protein multi-layering of the porous hollow-fiber membranes immobilizing the ionizable polymer brushes provides useful information on the spatial profile of the ionizable functional groups along the polymer brushes. Here, diethylamino or sulfonic acid group and 2-hydroxyethylamino or diol functional groups were adopted as the ionizable group and coexisting hydrophilic group, respectively. In addition, bovine serum albumin and hen-egg lysozyme were bound to the polymer brushes immobilizing diethylamino and sulfonic acid groups, respectively, in a permeation mode.

A porous hollow-fiber membrane, supplied by Asahi Kasei Corporation, Japan, was used as the trunk polymer for grafting. This hollow fiber had inner and outer diameters of 2 and 3 mm, respectively, with an average pore size of 0.4 μm and a porosity of 70%. Glycidyl methacrylate was purchased from Tokyo Kasei Co., and used without further purification. Hen-egg lysozyme (HEL) and bovine serum albumin (BSA) were purchased from Sigma Co. Other chemicals were of analytical grade and higher.

Preparation of Ionizable Polymer Brushes onto Pore Surface.

Four kinds of ionizable or ion-exchange polymer brushes, i.e., two kinds of anion-exchange polymer brushes and two kinds of cation-exchange polymer brushes, were immobilized onto a porous hollow-fiber membrane by radiation-induced graft polymerization and subsequent chemical modifications. The chemical modifications consist of successive functionalization: (1) introduction of ion-exchange functional groups, i.e., diethylamino and sulfonic acid groups, and (2) introduction of alcoholic hydroxyl functional groups, i.e., 2-hydroxyethylamino and diol groups. The diethylamino (DEA) and sulfonic acid (SS), 2-hydroxyethylamino (EA) and diol groups were introduced by ring-opening reactions of the epoxy group of the poly-GMA brushes with diethylamine, sodium sulfite, ethanolamine, and water, respectively. The reaction conditions are summarized in Table F.

TABLE F
Preparation conditions of four kinds of ionizable polymer
chains grafted onto a porous hollow-fiber membrane
(a) Graft polymerization of GMA by preirradiation technique

| | |
|---|---|
| Electron beam: | |
| Irradiation dose | 200 kGy |
| Atmosphere | N₂ atmosphere |
| Temperature | ambient |
| GMA grafting: | |
| GMA concentration | 10 vol % in methanol |
| Temperature | 313 K |
| Reaction time | 10, 13 min |

(b) Introduction of ionizable and coexisting hydrophilic groups

| | Concentration | Temperature [K] | Reaction time [h] |
|---|---|---|---|
| SS group | SS/IPA/water = | 353 | 3 |
| | 10/15/75 (w/w/w) | | |
| Diol group | 0.5 M H₂SO₄ | 333 | 3 |
| DEA group | 50 vol % in water | 303 | 24 |
| EA group | 100% | 303 | 24 |

The order variation of the successive functionalization after GMA grafting produced four kinds of porous hollow-fiber membranes immobilizing the anion- or cation-exchange polymer brushes: the resultant four kinds of the porous hollow-fiber membranes were referred to as DEA-EA, EA-DEA, SS-Diol, and Diol-SS fibers. The degree of GMA grafting was set at 150%. Both the degree of GMA grafting and conversion were determined by the weight gain via the reactions as described.

Permeability of Porous Hollow-Fiber Membranes Immobilizing Ionizable Polymer Brushes.

The porous hollow-fiber membrane effective length of 5 cm was positioned accordingly. Tris-HCl buffer (pH 8.0) and carbonate buffer (pH 9.0) were forced to permeate radially outward through the pores across the DEA-EA or EA-DEA fiber, and the SS-Diol or Diol-SS fiber, respectively, at a constant transmembrane pressure of 0.05 or 0.10 MPa at 298 K. Permeation flux was evaluated from the following:
permeation flux=(permeation rate)/(inside surface area of each hollow-fiber membrane)

Protein Binding During Permeation Through Pores.

Protein dissolved in the buffer was forced to permeate through the pores of the porous hollow-fiber membrane. BSA in Tris-HCl buffer and HEL in carbonate buffer were fed to the DEA-EA or EA-DEA fiber, and the SS-Diol or Diol-SS fiber, respectively. The effluent penetrating the outside surface of the porous hollow-fiber membrane was continuously collected with fraction vials. The protein concentration of each vial was determined from the measurement of UV absorbance as described. The equilibrium binding capacity, i.e., the amount of protein bound in equilibrium with the feed concentration, was evaluated from the following integration:

$$q = {\int }_{0}^{Ve} ( {C}_{0} - C ) ⅆ V / W$$

where C₀ and C are the protein concentrations of the feed and effluent, respectively. V and Vₑ are the effluent volume and effluent volume where C reached C₀. W is the weight of the porous hollow-fiber membrane in a dry state.

The permeation flux for the porous hollow-fiber membranes to immobilize the anion- and cation-exchange polymer brushes is a function of the conversion of the epoxy group into the corresponding ionizable group. The DEA-EA and EA-DEA fibers exhibited almost the same permeation flux below a conversion of 60%. Beyond this conversion the permeation flux of the DEA-EA fiber gradually decreased. On the contrary, the SS-Diol and Diol-SS fibers made a remarkable difference. Even at a conversion of 5% the SS-Diol fiber had a negligibly low permeation flux, whereas the permeation flux of the Diol-SS fiber maintained 40% of that of the original porous hollow-fiber membrane even at a conversion of 50%.

The DEA-EA fiber held BSA in multilayers over a conversion of 20%, whereas the EA-DEA fiber had a constant amount of bound protein equivalent to monolayer binding capacity. On the contrary, the SS-Diol fiber exhibited a high degree of multilayer binding of HEL at a lower conversion, whereas for the Diol-SS fiber the same conversion showing the degree of HEL multilayer binding as the SS-Diol fiber shifted to a higher value by approximately 20%. For example, the SS-Diol and Diol-SS fibers exhibited almost the same amount of adsorbed HEL of 80 mg/g at the conversion of 5 and 35%, respectively.

The order variation of successive chemical modifications of polymer brushes had an influence on the performance of the ionizable polymer brushes. This can be explained by a simple principle regarding the ionizable group distribution along the polymer chains grafted onto the porous hollow-fiber membrane. The first reagents for the functionalization attack the epoxy groups in the upper part of the poly-GMA chains, and the second reagents ring-open the remaining epoxy group in the lower part.

The poly-GMA chains grafted onto a porous hollow-fiber membrane, made of polyethylene, are formed in two domains because the radicals are uniformly produced throughout the polyethylene matrix by preirradiation with the electron-beam: (1) the polymer chains imbedded in the depth of the polyethylene matrix, and (2) the polymer chains extending from the pore surface toward the pore interior.

The polymer chains of the DEA-EA fiber consist of the DEA-group-rich upper region and the EA-group-rich lower region. When the conversion of the epoxy group into the DEA group exceeded the conversion of 20%, BSA was bound in multi-layers by the polymer brushes. Whereas, the polymer brushes of the EA-DEA fiber are not allowed to extend themselves from the pore surface toward the pore interior even at a conversion of 60% because the weakly ionizable EA groups are introduced into the upper region of the polymer brushes.

Even at a conversion of 5%, the SS-Diol fiber immobilizing the strongly ionizable polymer brushes reasonably exhibited a low permeation flux and a high degree of multilayer binding of HEL. Whereas, the performance of the Diol-SS fiber is governed by the character of the diol-group-rich upper region of the polymer brushes, and, nevertheless, beyond a conversion of 25%, the polymer brushes start to extend, resulting in the occurrence of multilayer binding of HEL.

The extension of the ionizable polymer brushes is governed by the internal parameters such as the length and ionizable-group density of the polymer brushes and the external parameters such as pH and ionic strength of surrounding liquids. This suggests a new parameter determining the degree of the extension of the polymer brushes—the order variation of successive functionalization for the epoxy-group-containing polymer brushes prepared by radiation-induced graft polymerization. The polymer brushes were appended onto the pore surface of the porous hollow-fiber membrane. The density of poly-GMA brushes amounted to 8 to 12 mol per kg of the porous hollow-fiber membrane. Thus, order variation of successive modifications provides for immobilization of functional groups along the surface of the brush in multi-layers.

### EXAMPLE FIVE

### Urea Hydrolysis Using Urease Immobilized in Multi-Layers onto Porous Hollow-Fiber Membranes

Urease was immobilized by ion-exchange polymer brushes grafted onto the pore surface of a porous hollow-fiber membrane with a porosity of 70% and a thickness of approximately 1 mm. The density of immobilized urease amounted to 1.6 gram per gram of the membrane. Urease bound in multi-layers by the polymer brushes via ion-exchange adsorption was crosslinked with transglutaminase. A 2 M urea solution was forced to permeate radially outward through the pores rimmed by the urease-immobilized polymer brushes at a constant permeation rate of 30 mL/h. The reaction percentage of urea hydrolysis increased to 100% at 310 K with an increase in the density of the immobilized urease. The reaction percentage of urea hydrolysis remained as high as 80% when the initial urea concentration was increased to 8 M.

Enzymes were multi-layered onto charged or ion-exchange polymer brushes grafted onto a porous hollow-fiber membrane at a high rate, for example, urease (pI 5.1) dissolved in Tris-HCl buffer (pH 8.0) was transported by convective flow to the vicinity of positively charged, i.e., anion-exchange, polymer brushes that extend themselves due to electrostatic repulsion. As much as 1.6 g of urease per gram of the membrane was bound to the polymer brushes.

Urease bound to the polymer brushes at a high density may be utilized in the efficient hydrolysis of urea. Here, crosslinking of the bound urease is required because urea hydrolysis forms ammonia and carbon dioxide to induce a pH change; some of the urease bound to the polymer brushes via electrostatic interaction or ion-exchange adsorption will be released from the polymer brushes. Moreover, a novel enzymatic system using the enzyme-immobilized porous hollow-fiber membrane has two distinct advantages: (1) high density of immobilized enzymes: enzymes are multi-layered by ionizable polymer brushes grafted onto the pore surface of the porous membrane because of electrostatic repulsion of ionizable brushes, and (2) high speed transport of substrates: the diffusion path of the substrate to the enzyme-immobilized brushes is minimized by convective flow of the substrate solution through the pores driven by transmembrane pressure.

The hydrolysis of urea at such high concentrations (2˜8 M) has not been reported thus far. A higher density of urease immobilized onto the brushes enables the efficient hydrolysis of a higher concentration of urea. The objectives of this study were two-fold: (1) to immobilize urease at various immobilized densities onto a porous hollow-fiber membrane, (2) to demonstrate the urea hydrolysis performance of urease-immobilized porous hollow-fiber membranes.

Preparation of Anion-Exchange Porous Hollow-Fiber Membranes

In order to bind urease based on electrostatic interaction, a diethylamino (DEA) group (—N(C₂H₅)₂) as an anion-exchange group was introduced into a porous hollow-fiber membrane. Briefly, an epoxy-group-containing vinyl monomer, glycidyl methacrylate was grafted onto an electron-beam-treated porous hollow-fiber membrane made of polyethylene. The degree of GMA grafting (dg) defined below was set at 150%. Some of the epoxy groups of the grafted polymer brushes were converted into the DEA group, and the remaining epoxy group were ring-opened with ethanolamine. The conversion of the epoxy group into the DEA group, defined above, ranged up to 80% by varying the immersion time of the GMA-grafted membrane in diethylamine. The resultant anion-exchange porous hollow-fiber membrane was referred to as a DEA(x)-EA fiber, where x designates the conversion.

Adsorption of Urease During Permeation Through the Membranes

The DEA(x)-EA fiber with an effective length of 1.2 cm was used to hydrolyse urea. One end of the hollow fiber was connected to a syringe pump and the other end was sealed. Urease solution, the concentration of which was 5.0 mg/mL of Tris-HCl buffer (pH 8.0), was permeated radially outward from the inside surface of the hollow fiber to the outside surface at a constant permeation rate of 30 mL/h at 310 K. The effluent penetrating the outside surface of the hollow fiber was continuously collected using fraction vials. Urease concentration in each vial was determined by measuring the UV absorbance at 280 nm. The amount of urease bound to the DEA(x)-EA fiber was evaluated as follows:
q(g/g)=∫₀^{Ve}(C₀−C)dV/W
where C₀ and C are the urease concentrations of the feed and the effluent, respectively. V, Vₑ, and W are the effluent volume, the effluent volume when C reaches C₀, and the mass of the DEA(x)-EA fiber in the dry state, respectively.

Immobilization of Urease via Crosslinking with Transglutaminase

In order to prevent the leakage of urease from the grafted polymer brushes, the urease-bound fiber was immersed in 0.04 wt % transglutaminase solution to crosslink urease. Subsequently, the hollow fiber was set again in the permeation mode. NaCl (0.5 M) was permeated radially outward through the hollow fiber to elute the non-crosslinked urease. Urease concentration of the effluent penetrating the outside surface of the hollow fiber was continuously determined. The amount of urease immobilized onto the hollow fiber was evaluated by subtracting the amount of eluted urease from the amount of bound urease. The resultant urease-immobilized porous hollow-fiber membrane was referred to as a Urease(q) fiber, where q designates the density of immobilized urease.

Determination of Activity of Immobilized Urease

Urea solutions of 2˜8 M were forced to permeate through the Urease(q) fiber at a constant permeation rate of 30 mL/h at 310 K. The effluent penetrating the outside surface of the Urease(q) fiber was continuously collected. Urea concentration of the effluent was determined using the diacetylmonoxime method. The pressure required to keep the permeation rate of the urea solution constant was measured.

This is because the grafted polymer brushes extend themselves more from the base material surface due to the higher degree of electrostatic repulsion induced by the increase in DEA group density.

By crosslinking with transglutaminase, approximately 80% of the bound enzyme was immobilized over the range of the amount of bound urease from 0.2 to 2.0 g/g. For example, at a conversion of 70% of the epoxy group into the DEA group, the density of enzyme immobilized onto the porous hollow-fiber membrane was 1.5 g of urease per g of the DEA-EA fiber. Properties of the Uase fiber described herein are summarized in Table G.

TABLE G
Properties of anion-exchange porous hollow-fiber
membrane for immobilization of urease.

| | |
|---|---|
| Degree of GMA grafting (%) | 150 |
| Functional group density (mmol/g) | |
| Diethylamino group | 2.3 |
| 2-hydroxyethylamino | 0.6 |
| Size (mm) | |
| Inner diameter | 2.0 |
| Outer diameter | 4.1 |

Urea Hydrolysis Using the Urease Fiber

Permeation of a sample solution comprising a substrate, i.e., urea, through the enzyme-immobilized porous membrane ensures a negligible diffusional mass-transfer resistance of the substrate from the bulk to the enzyme-immobilized polymer brushes; a higher density of immobilized enzyme will exhibit a higher activity of enzymes per unit mass of the supporting porous membrane. The reaction percentage in the hydrolysis of 2 M urea solution at 310 K is a function of the density of immobilized urease. The reaction percentage increased with an increase in the density of immobilized urease and leveled off above the density of 1.4 g of urease per g of the DEA-EA fiber.

The amount of urea hydrolyzed per unit mass of enzyme decreased with an increasing density of immobilized urease. This finding indicates that the diffusion of urea into the depth of the enzyme immobilized in multi-layers by the polymer brushes grafted onto the pore surface is a contributor to the overall hydrolysis rate of urea regardless of the negligible diffusional mass-transfer resistance of urea from the bulk to the interface between the bulk and the enzyme-immobilized polymer brushes.

At a contact time of 0.2 h, the increase of initial urea concentration decreased the reaction percentage of free enzyme rapidly from 100% (at 2 M urea concentration) to 40% (at 6 M urea concentration). On the other hand, the reaction percentage of the immobilized enzyme still maintained at more than 80% with an initial urea concentration of 8 M (residence time of 0.2 h).

Changes of urea reaction percentage and pH of the effluent as a function of effluent volume when a 8 M urea was permeated through the enzyme-immobilized membrane are observed. The pH and the reaction percentage remained unchanged even when the effluent volume was increased.

The diethylamino-group-containing polymer brushes were appended onto a porous hollow-fiber membrane made of polyethylene by radiation-induced graft polymerization of an epoxy-group-containing reactive monomer and subsequent reaction with diethylamine. The anion-exchange polymer brushes extended themselves from the pore surface of the porous hollow-fiber membrane due to electrostatic repulsion to bind enzymes in multi-layers. Urease was bound in multi-layers during the permeation of urease solution across the anion-exchange porous hollow-fiber membrane. The bound urease was crosslinked with transglutaminase to prevent the leakage of the enzyme induced by the pH change with the progression of urea hydrolysis. The density of immobilized urease was as high as 1.6 g of urease per g of the anion-exchange porous hollow-fiber membrane. Urea solutions (2˜8 M) were permeated through the urease-immobilized porous hollow-fiber membrane at a constant residence time of 12 sec at 313 K. While the activity per unit mass of immobilized urease decreased due to the diffusional mass-transfer resistance of urea into the multi-layered enzymes, the activity per unit mass of the urease-immobilized porous hollow-fiber membrane increased with an increase in density of the immobilized urease.

Hydrolysis percentage of urea using the Uase (1.2) fiber at a constant permeation rate of a urea solution of 1 mL/h is a function of a dimensionless effluent volume (DEV), defined by dividing the effluent volume by the membrane volume excluding the lumen part of the hollow fiber. The concentration of the urea solution fed to the inside surface of the Uase fiber ranged from 2 to 8 M. A permeation rate of 1 mL/h corresponded to a residence time of 5.1 min of the urea solution through the pore of the Uase fiber. A quantitative hydrolysis of urea at 2 and 4 M was achieved, and for 6 to 8 M urea the hydrolysis percentage gradually decreased with an increasing DEV. Hydrolysis percentage of 4 M urea by using Uase fiber is shown in FIG. 24 as a function of space velocity (SV) calculated by dividing the permeation rate by the membrane volume. At an SV of lower than 20 h⁻¹, i.e., a residence time of longer than 3.0 min, 100% hydrolysis of urea was observed; permeation rate of the urea solution to the Uase fiber governs the overall hydrolysis rate of urea. As SV increased, the hydrolysis percentage decreased; the overall hydrolysis rate of urea is determined by diffusion of urea in urease multilayered in the polymer chain and intrinsic reaction at the active site of immobilized urease.

### EXAMPLE SIX

### Preparation of a Protein Separation Tube

Preparation of a Protein Separation Tube

Another device of the present invention includes functionalized tubing. A Teflon® based tube (inner diameter 1 mm, length 10 cm) was irradiated by electron beam. The total dose of the applied electron beam was set at 20, 30 and 50 kGy. As described, the increase of total irradiation dose leads to the increase of polymer brush density. Glycidyl methacrylate (GMA) was grafted onto the lumenal surface of the tube. The degree of grafting of GMA was calculated as described. The epoxy groups of GMA were then converted into trimethylamine (TMA) groups using standard chemical reaction techniques. The tube with about 90% of TMA conversion was selected for further uses described herein.

Performance of The Ion-Exchange-Group-Containing Tube

Cl⁻ ion or bovine serum albumin (BSA) solutions were permeated through the inner part of the prepared TMA tube. The flow rate was set at 5 mL/h. The feed concentrations for BSA and HCl solutions were 0.05 g/L and 2.5 mM, respectively. The adsorption performance of the tube, i.e., its ability to immobilize chloride ions (Cl⁻) and bovine serum albumen (BSA), was measured as a function of degree of grafting and total irradiation dose.

The profile of the breakthrough curves for Cl⁻ and BSA respectively, are a function of the degree of grafting. The adsorption amount of Cl⁻ and BSA increased with the degree of grafting. The breakthrough curves of Cl⁻ reach 100% of the feed concentration even if the degree of grafting was increased, meaning that the adsorption has achieved equilibrium. In contrast, the adsorption of BSA increased gradually when the degree of grafting was increased.

When the total irradiation dose was held constant, the increase of degree of grafting resulted in the increase of the length of the poly-GMA brushes. Therefore, for a Cl⁻ ion, which is 1/10 of the size of BSA and has a small diffusion coefficient (200×10⁻¹¹), the diffusion time along the polymer brush is independent from the length of the polymer brush. However for BSA (diffusion coefficient=6.7×10⁻⁹) which is approximately ten times larger than the size of a Cl⁻ ion, the longer the polymer brush, the more time the BSA will need to diffuse into the brushes. As a result, the TMA tube of 2% of degree of grafting showed a more gradual adsorption of BSA.

When the total irradiation dose was varied, the density of the polymer brushes varied. For the Cl⁻ breakthrough curves, the Cl⁻ adsorption amount was constant irrespective of the total irradiation dose. This is due to the small size of the Cl⁻ ion. For BSA, the adsorption amount also increased with the increase of total irradiation dose. However, the BSA adsorption only reached equilibrium with the 50 kgy-irradiated TMA-tube. Without being bound to theory, the increase of the total irradiation dose led to the increase of brush density, making it difficult for the BSA to permeate into the brushes.

### EXAMPLE SEVEN

### Functionalized Materials with Specific Affinity for One or More Ligands

Pipet tips, tubing, ELISA plates, and porous hollow fiber membranes were irradiated by electron beams to initiate radical induced polymerization. The total dose of the applied electron beam was set at 20, 30 and 50 kGy. Glycidyl methacrylate (GMA) was grafted onto the lumenal surface. The degree of grafting of GMA was calculated as described.

Staphylococcus protein A (SpA) or Streptomyces Protein G (SpG), or the cellular receptor FcRn was immobilized to the polymer brush surface. These functionalized materials were then used to adsorb immunoglobulin from serum, ascites, and cell culture supernatants. The immunoglobulins were eluted from the functionalized materials by high ionic strength (approximately 0.5 M NaCl) buffers. Other elution conditions are possible and known to those skilled in the art. The eluted immunoglobulins comprised mixtures of isotypes, (i.e., IgG1, IgG2, IgG3, and IgG4 from human serum) polyclonal preparations (from the serum of antigenically challenged rabbits), monoclonal preparations-(from mouse ascites and from cultured hybridomas).

The immunoglobulin preparations were then immobilized on unused functionalized materials, which were used in the subsequent immunospecific purification and concentration of polypeptides for which the immunoglobulin molecules had specific affinity or avidity, i.e., the HIV gp120, protease and reverse transcriptase proteins, hemagglutinin, neuramimidase, IL-1, IL-6, TNF, peptidoglycan, CCR1, and the HER2 gene product. Antibodies to these proteins are also commercially available from a number of sources.

Whole immunoglobulin molecules can be used, including chimeric, humanized and bispecific antibodies, but the invention also permits immunoglobulin fragments to be used, i.e., Fab, F(ab)₂, Fv, and Fc domains or fragments. The immobilization of such fragments are within the capabilities of those skilled in the art.

### EXAMPLE EIGHT

### Functionalized Materials with Specific Affinity for One or More Ligands

PTFE conduit was prepared having trimethymamine (TMA) groups, imparting ion exchange functionality to the tubing materials. While tubing is illustrated as a non-limiting example, this technique has particular application to any polymer surface, and in a currently preferred embodiment, with materials defining a lumenal surface, i.e., pipet tips.

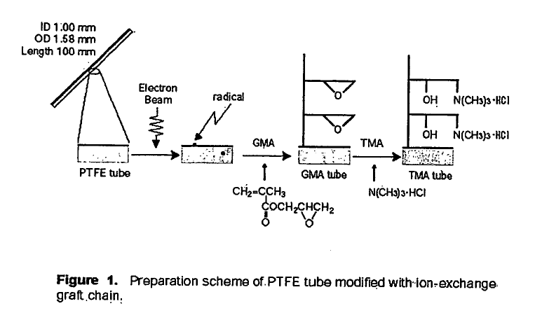

FIG. 1 shows the preparation scheme for the protein separation tube. Glycidyl methacrylate (GMA) was grafted onto the Teflon-based tube (inner diameter 1 mm, length 30 cm) after the irradiation of electron beam. The total dose of electron beam was set at 20, 30 and 50 kGy. The amount of GMA grafted onto the tube is defined as:
Degree of grafting=100 [(weight of grafted GMA)/(weight of base Teflon tube)]%
The epoxy group of the GMA was then converted into the trimethylamine (TMA) group.

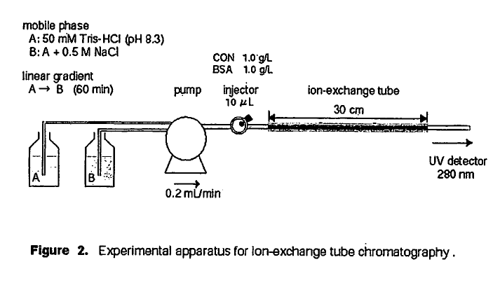

Conalbumin (CON) or bovine serum albumin (BSA) solutions were permeated through the inner part of the prepared TMA tube of 30-cm long (FIG. 2). The separation performance of CON and BSA was measured as a function of apparent graft chain density. The flow rate was set at 0.2 mL/min. The feed concentration for both CON and BSA was 1.0 g/L.

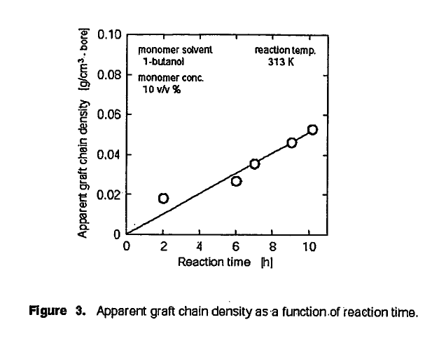

FIG. 3 shows the apparent graft chain density as a function of grafting reaction time. The concentration of GMA is 10 vol/vol % in 1-butanol and the grafting reaction temperature is 313 K. The apparent graft chain density (g/cm³-bore) increased with increasing reaction time.

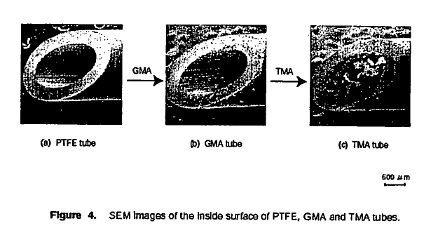

FIG. 4 shows the scanning electron microscopy (SEM) of the inside surface of PTFE, GMA and TMA tubes.

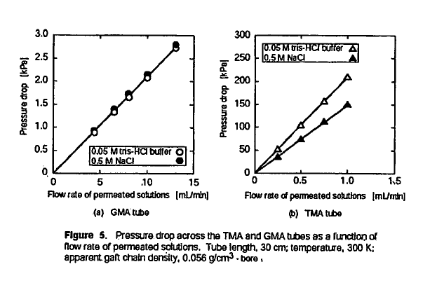

FIG. 5 shows the pressure drop across the (a) GMA and (b) TMA tubes as a function of flow rate of permeated solutions (0.05M Tris-HCl buffer and 0.5M NaCl). The length of both tubes is 30 cm and the permeation was made at 300 K The apparent graft chain density is 0.56 g/cm³-bore. For the GMA tube, the pressure drop for the Tris-HCl buffer is identical to the NaCl solution. However, the pressure drop of Tris-HCl buffer is higher than the NaCl solution for the TMA tube due to the extension and retraction of ionic graft chains, respectively.

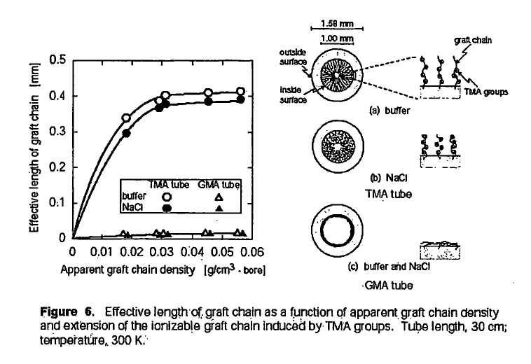

FIG. 6 shows the effective length of graft chains as a function of apparent graft chain density and the extension of the ionizable graft chain induced by the TMA groups. As illustrated, the introduction of TMA groups on the graft chains has ionized the graft chains and led to the extension of the graft chains by the repulsion of electrostatic charges on the graft chains. However, for the GMA tube, due to the neutral epoxy groups, the graft chains were not able to extend their arms in both buffer and salt solutions.

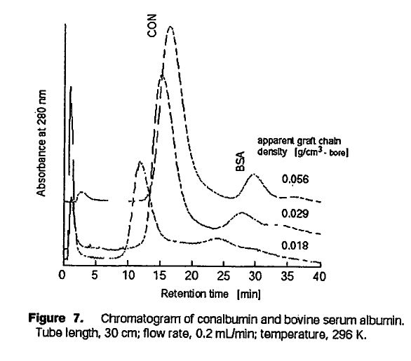

FIG. 7 shows the chromatograms for the separation of conalbumin (CON) and bovine serum albumin (BSA). The TMA has showed its ability to separate CON and BSA and the resolution increased with the increasing apparent graft chain density.

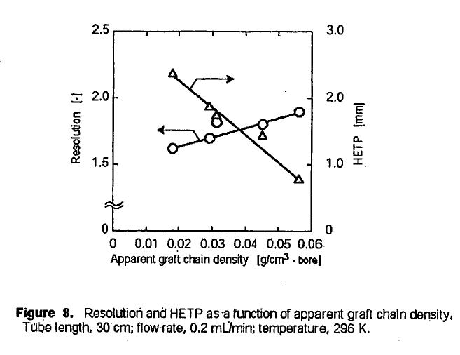

FIG. 8 shows the resolution and HETP (height equivalent to theoretical plate) as a function of apparent graft chain density. The better HETP performance is achieved with better resolution when the apparent graft chain density is above 0.04 g/cm³-bore.

### EXAMPLE NINE

### Immobilization of Protein A and Protein G to the Functionalized Materials

The bacterial proteins Staphyloccocus protein A (SpA) and Streptomyces protein G (SpG) provide functional groups that are well suited to affinity purification of mammalian immunoglobulins, for example, murine, rat, rabbit, goat, and human IgG. The following example details immobilization of SpA to the base materials, in this case a hollow fiber, resulting in functionalized materials capable of binding immunoglobulins. Other base materials such as pipet tips, tubing and ELISA plates are useful, and prepared as described throughout the specification.

While SpA is described, SpG is immobilized in as described, and offers the advantage of binding a broader range of isotypes as well as immunoglobulins from a wider variety of species. Purified SpA and SpG were used for these studies, but a common technique for immunoglobulin precipitation is to use preparations of killed and formaldehyde-fixed bacteria expressing these Ig binding proteins on their cell surfaces, i.e., Staphyloccocus aureus. The materials of the present invention are suitable for using such microorganisms as Ig binding functional groups, as the polymer brush surface permits entrapment of the microbe, which is then cross-linked to the brush surface.

(1) Preparation of Functionalized Membrane

A porous polyethylene hollow-fiber membrane was used as the trunk polymer for grafting. This hollow fiber had inner and outer diameters of 2 and 3 mm, respectively, with an average pore size of 0.4 μm and a porosity of 70%. A vinyl monomer, glycidyl methacrylate (GMA, CH₂═CCH₃COOCH₂CHOCH₂) was grafted onto the PE membrane by applying the electron-beam-induced grafting method. The PE membrane was irradiated with an electron beam at a total dose of 200 kGy at room temperature. The irradiated membrane was then immersed in a GMA solution (10 vol/vol % in methanol) and reacted at 313 K. The amount of GMA grafted onto the backbone membrane was described as the degree of GMA grafting (DG)
DG(%)=100 (weight increase due to grafting)/(weight of the trunk polymer)
Here, the DG was set at 160%. The resultant GMA-grafted porous hollow-fiber membrane was referred to as the GMA membrane.
(2) Immobilization of Protein A onto the GMA membrane

A GMA membrane of 2.5-cm long was immersed in a 1.7 mg/mL Protein A (molecular weight 42,000) solution for 16 h at 277 K. The amount of Protein A immobilized was measured using a spectrophotometer (at 280 nm) and calculated from the concentration difference of the Protein A solution.

Results:

The amount of Protein A immobilized (or coupled) onto the functionalized GMA membrane (amount of GMA: 0.3 mmol) was 4.9×10⁻⁶ mmol. This figure is equal to a Protein A density of 0.07 μmol/g-membrane or about 3 mg Protein A/g-membrane or 7.0 mg Protein A/mL-membrane. The optimal quantity of antibody that can be adsorbed by this membrane can be approximated by assuming a 1:1 molar ratio between the weight of immobilized SpA and the immunogobulin.

The SpA or SpG immobilized membranes were used to adsorb the following monoclonal and polyclonal immunoglobulin preparations: anti-v-H-Ras, anti-Rb, anti-v-Src, anti-c-Ab1, anti-beta amyloid, anti-Bcl-2, anti-BRCA1, anti-C3, anti-Caspase-1, anti-CD4, anti-CD8, anti-CD40, anti-CD40L, anti-CD56, anti-EBNA, anti-EGF, anti-estrogen receptor, anti-alpha interferon, anti-gamma interferon, anti-IL-6, anti-JAK1, anti-c-Jun, anti-MAP kinase ERK2, anti-c-Myc, anti-NF-kappaB, anti-p53, anti-PDGF, anti-PI3 Kinase, anti-PSA, anti-PKA, anti-PKC, anti-TGF-beta, and anti-VEGF (all commercially available through Calbiochem, La Jolla Calif., respectively having catalog numbers OP01A, OP28A, OP07A, OP20, 171603, OP60, OP92, 204869, PC84, 217575, 217580, 217590, 217595, 217671, DP15L, PCO8, 330659, 407295, 407309, 407670, 420092, PC06L, 442680, OP10, PC136, OP43, PC21, 528107, OP158, 539231, OP74, PC193L, and PC36). These can in turn be immobilized to the polymer brushes by cross linking to impart an additional leval of functionality to the membrane. For example, a membrane with SpG and anti-hemagglutinin immobilized thereto is able to purify HA-epitope tagged recombinant fusion polypeptides from bacterial cruse lysates. Often this provides better results than immobilizing the antibody directly to the brush surface, for example through its epoxy groups. Without being bound by theory it is believed that immobilizing the immunoglobulin by first binding it to SpA or SpG does not distort the structure of the immunoglobulin, i.e., the immobilization occurs at the CH2/CH3 region and the complementarity determining regions (e.g., the Fab domain) is not appreciably affected.

### Equivalents

From the foregoing detailed description of the specific embodiments of the invention, it should be apparent that a unique compositions comprising graft polymerized materials having functional groups immobilized thereto in multiple layers, as well as methods of making and using such compositions, has been described. Although particular embodiments have been disclosed herein in detail, this has been done by way of example for purposes of illustration only, and is not intended to be limiting with respect to the scope of the appended claims that follow. In particular, it is contemplated by the inventor that substitutions, alterations, and modifications may be made to the invention without departing from the spirit and scope of the invention as defined by the claims. For instance, the number and kind of functional group combinations, or the use of such compositions in particular devices is believed to be matter of routine for a person of ordinary skill in the art with knowledge of the embodiments described herein.

We claim:

1. A conduit comprising an interior surface and an exterior surface, the interior surface defining a lumenal space, and the interior surface having grafted polymer brushes further comprising functional groups.

2. The conduit of claim 1, wherein the functional groups are disposed along the brush surface in a plurality of layers.

3. The conduit of claim 1, wherein the functional groups are selected from the group consisting of hydrophilic groups, hydrophobic groups, anionic groups, enzyme groups, cationic groups, affinity groups and a combination thereof.

4. The conduit of claim 3, wherein a solution is passed through the lumen of the conduit and molecules in the solution are adsorbed to the functional groups thereby reducing the concentration of the molecules in the solution as the solution is passed through the conduit.

5. The conduit of claim 3 wherein a solution is passed through the lumen of the conduit and molecules in the solution react with the functional groups thereby reacting with the molecules in the solution as the solution is passed through the conduit.

6. A methods of using the conduit of claim 4 to remove nucleic acids from the solution.

7. A method of using the conduit of claim 4 to remove polypeptides from the solution.

8. A method of purifying a solution comprising passing a solution through the conduit of claim 4.

9. A method of purifying a solution comprising passing a solution through the conduit of claim 5.

10. A method of separating the molecules in a solution comprising passing the solution through the conduit of claim 4, thereby separating the molecules in the solution as the solution is passed through the conduit.

11. A method of separating the molecules in a solution comprising passing the solution through the conduit of claim 5, thereby separating the molecules in the solution as the solution is passed through the conduit.

12. A method of making a functionalized conduit comprising obtaining a conduit comprising an interior surface and an exterior surface, the interior surface defining a lumenal space, grafting polymer brushes to the interior surface, and immobilizing functional groups on the surface of the brushes.
